(19) United States
(12) Patent Application Publication
(10) Pub. No.: US 2006/0057554 A1
(43) Pub. Date: Mar. 16, 2006

(54) Sample collecting device and mass spectrometry of device

(76) Inventors: Roger John Watling, Western Australia (AU); Hugh Keith Herbert, Queensland (AU)

Correspondence Address: EDWARDS & ANGELL, LLP, P.O. BOX 55874, BOSTON, MA, 02205, US

(21) Appl. No.: 10/511,505

(22) PCT Filed: Apr. 16, 2003
(86) PCT No.: PCT/AU03/00450

§ 371 Date: Oct. 14, 2004

(30) Foreign Application Priority Data
Apr. 16, 2002 (AU) PS 1772

(51) Int. Cl. C12Q 1/00; C12M 1/34

(52) U.S. Cl. 435/4; 435/287.2; 436/86

(57) ABSTRACT

A sample collection device comprising a support bearing an inert absorbing matrix for a fluid sample is described. The device may or may not have a lancet. Also described for a sample device is a method of using a mass spectrometer in a laboratory where the sample in its matrix is ionised and the plurality of elements is detected. The results may or may not be quantised in relation to the original sample and an internal ionised reference sample may also be used.

### TECHNICAL FIELD

The present invention is concerned with methods and devices for sample collection and simultaneous detection and/or quantitation of multiple trace elements in fluid samples.

### BACKGROUND ART

A wide range of trace metals and other elements is necessary for good health and physical well being in humans and other animals; deficiencies in essential elements have been shown to cause general malaise and lead to the induction of specific disease, commonly resulting in death. For many essential trace elements, it is not simply the absolute concentration, but also the inter-element balances that have a profound effect on health. For example, selenium deficiency is implicated in the aetiology of Iodine Deficiency Disorders amongst humans, whilst copper deficiency, associated with high levels of manganese, may be implicated as a predisposing or causative factor in induction of Bovine Spongiform Encephalopathy (BSE) in cattle and, by association, New Variant Creutzfeldt-Jakob Disease (nvCJD) in humans.

Dietary forages, vegetables, grains and fruits, which fix available trace elements as metal colloids within their tissue, have long been regarded as sources of essential trace elements. Such plant-based metal colloids are about ninety-eight percent absorbed and communities and animals that have a balanced range of plant products as essential components of diet may reasonably be expected to display markedly reduced incidence of specific trace element deficiency-related disease when compared with other groups lacking quality forage or a regular vegetable, fruit and grain intake.

The trace element content of vegetative material is directly related to the bioavailability of essential nutrients in soils supporting the vegetation. Soils vary in their trace element content from enriched to impoverished, according to local geology, soil degradation and nutrient impoverishment and as a function of inappropriate cropping practice, which is widespread throughout the world. In addition, soils throughout the world are sustaining increasing anthropogenic chemical damage threatening the existence of many plants and animals. Consequently, human health is being threatened through the food chain.

While the productivity of the soils may be maintained through the application of N-P-K fertilisers, food crops growing on these soils becomes, without the regular application of biologically-available ‘balanced’ trace elements, progressively impoverished in essential trace elements and minerals. If not corrected, this may result in sharply increased incidences of mineral deficiency-related disease.

Elements may be classified as being essential or toxic to human and animal health. In the case of animals, trace metal deficiency and/or toxicity is due largely to concentration levels controlled by environmental factors, whereas for humans, both environmental and occupational factors may be important; toxic response may a function of both natural and/or anthropogenic influences.

Ignoring carbon, hydrogen and oxygen, the biologically essential major elements are calcium, chlorine, magnesium, phosphorous, potassium, sodium, nitrogen and sulphur. Essential trace elements include bromine, chromium, cobalt, copper, fluorine, iodine, iron, manganese, molybdenum, selenium, silicon and zinc. If bio-available, many of these essential trace elements induce toxic responses, at elevated levels, or it out of balance with synergistic and/or antagonistic elements. Several other elements (lithium, scandium, rubidium, lanthanum) are minor essential elements.

In addition to dietary trace metal deficiency-induced disease, other cohorts of individuals are occupationally or environmentally exposed to a range of toxic element pollutants, which similarly induce general malaise and/or specific clinical symptoms commonly resulting in complications and death. Notable amongst these are arsenic, lead and mercury, which constitute the top three most hazardous substances on the US Environmental Protection Agency's Toxic Substances and Disease Registry priority list.

The leaching of heavy metals into the aquatic environment, and uptake by wildlife in the food chain, may have a profound impact on human health. Cadmium and mercury, in particular, are strongly bio-accumulated in fish and shellfish.

Although it is not possible to quantify the hazards and deleterious effects associated with all trace elements, some elements clearly present a more serious problem than others. Respectively ranked 1, 2, 3 and 7 on the NPL, arsenic, lead, mercury and cadmium, as elemental pollutants, are considered extremely toxic and the health effects of these elements have received a great deal of attention from research workers. Other elements on the list, in alphabetical order, are aluminium, antimony, barium, beryllium, chromium, cobalt, copper, manganese, nickel, plutonium, radium, selenium, silver, thallium, thorium, tin, uranium, vanadium and zinc

Unlike many essential trace elements, the concept of a therapeutic index cannot be applied to toxic elements such as lead, cadmium, mercury and arsenic. These toxic elements play no known role in metabolism, as no enzyme has been identified which specifically requires any of them as cofactors. They are extremely hazardous to life and, resulting from ingestion, have been involved in historic poisoning episodes of both human and animal populations. They are increasing in concentration in both aquatic and terrestrial environments due to anthropogenic inputs, and thus will continue to be a concern to toxicologists and clinicians.

Hence, proactive intervention to identify trace metal and element aberrations within general populations, thereby enabling the early implementation of targeted remedial strategies with consequent minimization of the huge social impact of trace metal-induced disease, is essential. However, mass screening of general populations for trace metal deficiencies and/or toxic metal excesses, with reference to age, sex, socio-economic status and physical geography, while acknowledged as being highly desirable in terms of preventative medicine, is presently impractical. So too, is the mass screening of human food chain components, such as slaughter animals, prior to their entering the food chain.

Present test methodologies require relatively large volumes of fluid samples (for example, 5-10 ml of blood) and are commonly trace element specific, that is, simultaneous measurement of other trace elements potentially present is not possible. Because of this, other relevant trace metals are either overlooked or require further fluid samples for their determination. In the case of blood, this involves invasive, often traumatic extraction, particularly for young children, babies and the elderly, using hypodermic syringes. The derivative body fluid products require stabilisation and preservation, and having regard for transmissible disease such as HIV, appropriate biohazard handling and disposal. Further, the large volumes required give rise to handling and storage problems.

There is no current technology available that can conveniently be used for the collection and broad-spectrum analysis of the trace element content of large numbers of blood and other body fluid samples. Presently available testing methods are cumbersome and expensive, placing the service outside the reach of the general population, particularly in underdeveloped regions where problems are often greatest. Further, there are no convenient and sensitive mass spectrometric methods for detecting pollutants or contaminants in fluids such as water or lubricants.

There is therefore a need for improved methodologies which will enable more efficient and cost effective screening of trace elements in fluid samples.

It is an object of the present invention to alleviate at least some of the disadvantages of prior art methods, or to provide a useful alternative.

### SUMMARY OF THE INVENTION

According to a first aspect there is provided a sample collection device comprising an inert collection matrix capable of adsorbing or absorbing a fluid sample, and a solid support, wherein the inert matrix is affixed to an area of the solid support

Particularly useful matrices may be selected from aragonite, aluminium hydroxide, titania, glucose, Starch “A”, Starch “B”, glucodin, cellulose powder/granules, fibrous cellulose, hydroxy butyl methyl cellulose, vegetable flour and the like, or mixtures thereof. Particularly preferred is fibrous cellulose. The fibrous cellulose matrix may be modified by oxidation and/or acid hydrolysis to improve its properties and thus provide enhanced reproducibility and sensitivity.

The vegetable flour may be selected from rice, maize, wheat, soy, rye or corn flour, or mixtures thereof. Particularly preferred is rice flour.

The inert matrix may also contain, on or within, one or more pre-calibrated selected analytes as internal standard, to aid in the quantitation of trace elements in the sample applied to the collection device.

The device of the present invention may also comprise an integral lancing member, capable of piercing for example skin or tissue, to aid in the collection and application of a blood or body fluid sample to the inert matrix. The lancing member may be mounted adjacent to, within or below the area of inert matrix. There may be included a guiding channel in the inert matrix, to guide the lance should it be disposed below the inert matrix area.

The device may also be equipped with a laser-scannable bar code which may contain patient information or other information concerning the sample, its nature and source. The device may also include an antibiotic barrier, to prevent contamination of the sample to analytical equipment and personnel.

Preferably the inert matrix is applied to only one side of the support. It is also preferred that the area to which the matrix is applied is smaller than the area of the solid support and that it be in the shape of a small tablet-sized disc.

The inert matrix may include hydrophobic and/or hydrophilic components, depending on the nature of the sample and the analysis to be performed.

Preferably the solid support is made of flexible material having sufficient durability to withstand transport and handling. Of course it will be understood that the support can be made of rigid material, depending on the nature of application. It is also preferred that the device is of sufficiently small size to allow transport of the device through mail and for ease of storage. The device may have an integral or separate cover sheath, to protect the inert matrix and prevent possible contamination after collection. The cover sheath also protects the device during transport and handling.

According to a second aspect there is provided a sample collection device having multi-layer construction wherein the collection matrix layer is sandwiched between two supporting layers, one of said supporting layers having an opening, which exposes an area of the collection matrix.

Alternatively, the sample collection device may encapsulate a collection matrix tablet within the body of the support wherein the matrix is exposed flush with one surface of the support.

The collection device and methods of the present invention may be used for analysis of any fluid sample, including body fluids, oils and other lubricants, water from drinking supplies as well as waste water, and the like. Body fluids such as whole blood are particularly preferred, however, separated blood (eg. plasma or serum) and other body fluids, such as urine or sweat, can also be used with the same device.

It will be understood that a sample of body fluid, particularly blood, can be collected for analysis by conventional means, or by using for example a sample collection kit comprising a resealable, sterile sample collection device, embodying a bar coded support in which is embedded, or to which is affixed, a tablet, wafer, wad, strip or the like, of sample absorption/adsorption matrix, a sealed alcohol-saturated wipe, and a separate retractable, single use, spring loaded lance for penetrating the skin and drawing blood. Of course a lance can be omitted from the kit if the sample to be collected is for example urine or sweat.

As indicated above, the analytical sample need not be a body fluid. Thus, the devices and methods of the present invention are equally applicable to collection and analysis of water or oil samples without significant adaptation of collection devices or analytical procedures and equipment.

The matrix of the sample collection device can include one or more matrix-matched standards either adsorbed/absorbed onto/into sample collection matrix or, alternatively, supported on an impermeable substrate. Here, the matrix may be spiked with elements, for example, Be, In and Hf and these elements will serve as internal standards that will be released simultaneously with the sample during ablation; this will facilitate matrix matching.

According to a third aspect there is provided a method of detecting simultaneously a plurality of elements in a fluid sample adsorbed onto or into an inert collection matrix, comprising:
(i) exposing the sample to high energy radiation capable of ionising at least a portion of the sample, and
(ii) detecting plurality of elements in the ionised portion of the sample by mass spectrometry.

According to a fourth aspect there is provided a method of quantifying simultaneously a plurality of elements in a fluid sample adsorbed onto or into an inert collection matrix, comprising:
(i) exposing the sample to high energy radiation capable of ionising at least a portion of the sample;
(ii) measuring quantity of a plurality of elements in the ionised portion of the sample by mass spectrometry;
(iii) measuring quantity of ionised portion of sample, and
(iv) determining quantity of the plurality of elements in the sample.

According to a fifth aspect there is provided a method of quantifying simultaneously a plurality of elements in a fluid sample adsorbed onto or into an inert collection matrix having an internal standard applied thereto, comprising:

(i) exposing the sample to high energy radiation capable of ionising at least a portion of the sample and a portion of said internal standard;
(ii) measuring quantity of a plurality of elements in the ionised portion of the sample by mass spectrometry;
(iii) measuring quantity of ionised internal standard in the ionised portion of the sample by mass spectrometry, and
(iv) determining quantity of the plurality of elements in the sample with reference to quantity of ionised internal standard.

According to a sixth aspect there is provided a method of quantifying simultaneously a plurality of elements in a fluid sample adsorbed onto an inert collection matrix comprising:
(i) introducing into the fluid sample a known quantity of a measurable internal standard
(ii) exposing the sample to high energy radiation capable of ionising at least a portion of the sample and the internal standard;
(iii) measuring quantity of a plurality of elements in the ionised portion of the sample by mass spectrometry;
(iv) measuring quantity of ionised internal standard in the ionised portion of the sample by mass spectrometry, and
(v) determining quantity of the plurality of elements in the sample with reference to quantity of ionised internal standard.

According to a seventh aspect there is provided a method of quantifying simultaneously a plurality of elements in a fluid sample adsorbed/absorbed onto or into an inert collection matrix comprising:
(i) exposing the sample to high energy radiation capable of ionising at least a portion of the sample;
(ii) measuring quantity of a plurality of elements in the ionised portion of the sample by mass spectrometry;
(iii) exposing a matrix-matched Certified Reference Material (CRM) to high energy radiation capable of ionising at least a portion of the CRM;
(iv) measuring quantity of ionised CRM in the ionised portion of the sample by mass spectrometry, and
(v) determining quantity of the plurality of elements in the sample with reference to the CRM.

According to an eighth aspect there is provided a method of quantifying simultaneously a plurality of elements in a fluid sample supported on an impermeable substrate, comprising:
(i) exposing the sample to high energy radiation capable of ionising at least a portion of the sample;
(ii) measuring quantity of a plurality of elements in the ionised portion of the sample by mass spectrometry;
(iii) exposing a matrix-matched Certified Reference Material (CRM) to high energy radiation capable of ionising at least a portion of the CRM;
(iv) measuring quantity of ionised CRM in the ionised portion of the sample by mass spectrometry, and
(v) determining quantity of the plurality of elements in the sample with reference to the CRM.

Details of some useful CRM's, for example, SARM 1, 3 and 46 (South African Bureau of Standards), and SY-2 (Canadian Certified Reference Material Project (CCRMP)) are given in Table 1. Other standard element cocktails may include elements such as Be, In, Hf, Bi, Th to cover the mass calibration range, but may include any element as a standard, that is not being analysed.

Preferably, the sample is whole blood and sample size is approximately 50 μl to 100 μl and even more preferred size of sample is 50 μl or less. Of course, separated blood may also be used, eg. plasma or serum.

Also preferred is that the high energy radiation is UV laser radiation and that the sample is exposed to such radiation for a period of approximately 30 seconds, but may be between 10 and 120 seconds. The devices and methods of the present invention may be used in conjunction with any inductively Coupled Plasma-Mass Spectrometer (ICP-MS) system. Particularly preferred are quadrupole and Time-of-Flight (TOF) ICP-MS systems.

The preferred elements to be detected and/or quantified are dietary trace elements, toxic elements and markers of pollution or wear and tear. For blood and other body fluids, these elements can include Li, Na, Mg, Al, P, K, Ca, Ti, V, Cr, Mn, Fe, Co, Ni, Cu, Zn, Ga, As, Se, Rb, Sr, Mo, Cd, Sn, Sb, Te, Ba, La, Ce, Eu, Dy, Yb, Hg, Tl, Pb, Th and Pb. For wear metals in lubricants such as oil, the element array may include Li, B, Mg, Al, Si, P, Ca, Tl, V, Cr, Mn, Fe, Co, Ni, Cu, Zn, Ga, As, Se, Sr, Y, Zr, Mo, Ag, Cd, Sn, Sb, Ba, La, Ce, Hf, Hg, Pb, and U.

In a preferred embodiment the matrix or the support comprise one or more wells or indentations to accommodate the fluid sample.

According to a ninth aspect there is provided a method of collecting a fluid sample for mass spectrometry analysis of multiple element content comprising the application of the sample to an inert matrix having a low background element content, wherein the matrix is selected from the group consisting of aragonite, aluminium hydroxide, titania, glucose, Starch “A”, Starch “B”, glucodin, cellulose powder/granules, fibrous cellulose, hydroxy butyl methyl cellulose, vegetable flour or mixtures thereof.

### DESCRIPTION OF THE PREFERRED EMBODIMENT

The present invention is in part based on Laser Ablation-Inductively Coupled Plasma-Mass Spectrometry technique, which allows rapid, automated, cost effective mass screening of general populations, bloodstock, zoo animals, pet and slaughter animals to identify trace element aberrations in body fluids. This technology facilitates proactive remedial intervention to target and correct essential trace element imbalances and/or toxic heavy metal excesses and enables identification and rejection of heavy metal-contaminated slaughter animals designed for human consumption. The methods and devices of the present invention are also useful for detection and quantitation of trace elements, metals and the like in fluids such water and lubricants, as indicators of for example water pollution or mechanical wear and tear.

The present invention in its various embodiments allows the simultaneous analysis and/or quantitation of a broad spectrum of up to 50 trace elements during a primary analytical run. A secondary run, using a screened torch may include Ca, Mg, Na, K and Fe. The analytical cost of a sample is lower than that of a large number of single element analyses currently being performed, on a chemically unmodified 50-100 micro-litre volume of body fluid sample or other fluid sample (single drop) adsorbed onto an inert collection matrix. In case of blood, the sample collection device, and collection protocol, may be so configured to eliminate the use of hypodermic syringes, and hence potential for stick injuries, is non-invasive and hence, non-traumatic, and does not involve the preservation, movement and storage of large volumes of blood and urine, or involve large biohazard disposal facilities. Indeed, in the case of humans, samples may generally be self-acquired at any geographic location through absorption/adsorption of a drop of biological fluid, such as blood from a pin prick, into/onto a lightweight collection device as described herein, and dispatched to the nearest analytical facility by post or courier. Because an approximately 8000° C. argon plasma is involved in ionisation of the samples, the body fluid samples are expected to be largely sterilized during analysis.

Certain embodiments of the present invention have been developed using an ultraviolet laser and quadrupole inductively coupled plasma-mass spectrometer (LA-ICP-MS) with manual sample handling. However, the present methods are equally applicable to Time-of-Flight (ToF) and High Resolution mass spectrometry techniques. Further, the methods of the present invention, whether they make use of quadrupole, ToF or High Resolution mass spectrometry, can be automated to allow rapid, high volume throughput screening of samples.

The methods and devices of the present invention permit cost effective, simultaneous, automated mass screening of blood, and other body fluids, for a wide range of essential and toxic trace elements on micro-litre volumes of test fluid absorbed onto inert collection matrices. In certain preferred embodiments the core of the analytical system comprises a quadrupole Laser Ablation-Inductively Coupled Plasma-Mass Spectrometer. The spectrometer may be used in conjunction with an associated automated sample insertion system.

In preferred embodiments of the present invention the collection device, or kit of parts, is envisaged to consist of the following components:
housing mount that forms the surround of the actual collection matrix and acts as the support of this matrix and also increases robustness of the entire device allowing for transport of the entire system;
the collection matrix itself consisting of an absorptive pellet;
a mechanism for puncturing skin and facilitating the collection of a single drop of blood; and
a bar code or equivalent which ultimately will facilitate the recognition of both the sample and its association with the client.

However, the collection device, or kits of parts, may exclude certain features or include additional features.

The invention will now be described in more detail with reference to non-limiting examples.

### EXAMPLES

### Example 1

### Sample Collection and Application

Samples may be collected and applied to a chosen collection matrix of the present invention in a conventional manner well known in the art.

For example, blood from a subject may be collected using a kit which comprises a shielded, retractable, spring loaded ‘pricker’, as part of the sample kit, which also includes a sealed, alcohol-saturated wipe, or swab, for pre-cleaning the skin area to be pricked to avoid unnecessary sample contamination.

It will be understood however that collection of samples of other body fluids, such as urine and sweat, or other fluids such as water or oil and other lubricants, will not require most of the components stipulated above for blood collection, but it will nevertheless be important to exclude contaminants. Conventional techniques for this will be known to those skilled in the art.

The fluid sample, which ever fluid may be of interest, can be applied to the collection matrix for analysis by any known means. For example, a particular quantity may be applied to the collection matrix by a pipette, a capillary tube, a dip-stick or similar device. Exact quantity applied is not important but may be controlled if desired.

Alternatively, particularly for blood sample collection, a collection device such as described in Example 2 below may be used.

### Example 2

### Sample Collection Device

An example of one type of sample collection device of the present invention, particularly suitable for collection of a blood sample, incorporates an inert fluid absorption matrix, most preferably a fibrous cellulose matrix (Whatman 540, but also 541, 542 and other cellulose filter papers, Whatman international Ltd, Maidstone, England), typically shaped in the form of a small tablet-size disc. The matrix is affixed to or encased within a small, lightweight, disposable or re-cyclable holder (disc holder or solid support material). Ideally the holder is made of relatively rigid material (for example plastic, cardboard or similar material). The device is designed so that a drop of blood or body fluid can be placed on the absorption matrix and the device sealed at the site of collection. Thus immobilized sample can be easily transported via post or courier to a sample analysis center and/or stored.

Of course the device may be used for other samples, which are not body fluids. For example water or a lubricants.

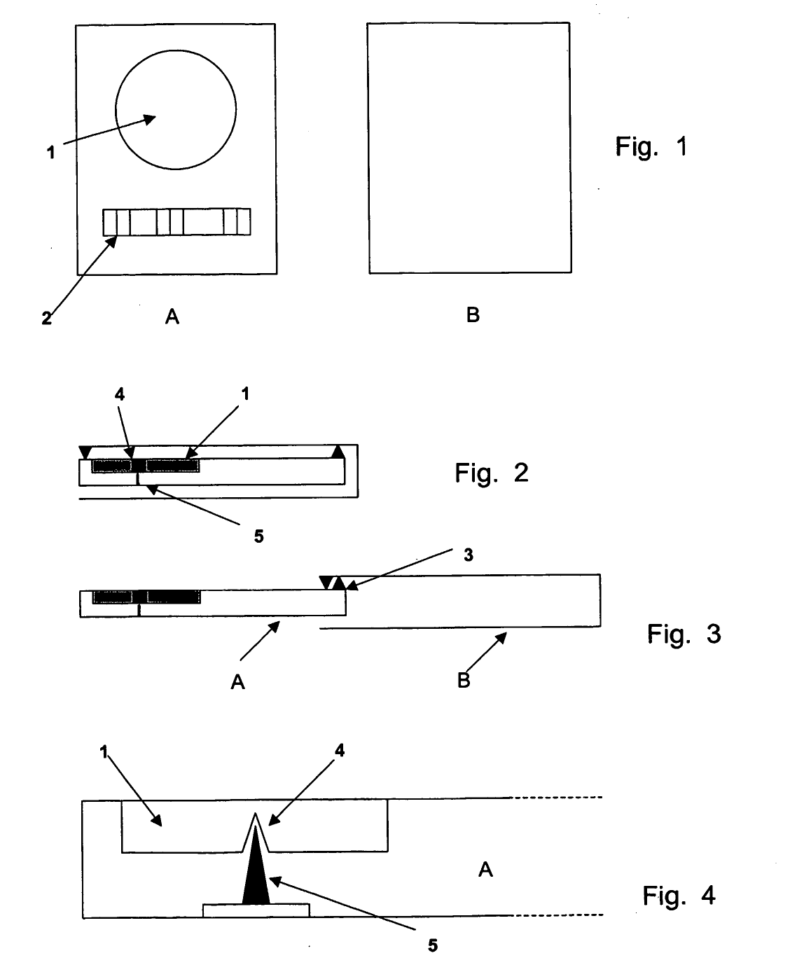

A collection device of this embodiment of the present invention, incorporating a number of features described below, is depicted in FIG. 1. In plan view (A) the device is typically rectangular in shape and has an area of absorbent collection matrix (1) disposed on the surface, and may also have a bar code (2) containing relevant information about the sample and/or the subject. The collection matrix is preferably fibrous cellulose but other matrices described hereafter may also be used. The collection area shown is circular in shape but may be any other suitable shape. A cover sheath (B) may be provided, to cover the collecting matrix area after the sample has been collected. FIGS. 2 and 3 show the collection device in cross section, in dosed and open positions respectively. The carrier or backing (support) portion (A) of the device can be suitably made of plastic or some form of card (stiff paper, cardboard and the like) material. The cover sheath (B) may be made of similar materials. Both the backing portion and the cover sheath may include a locking ridge (3), for positive engagement between the backing and cover sheath, and also to prevent the cover sheath, if used, from sliding off entirely.

FIGS. 2 and 3 also show the area of collection matrix (1) and a stylus or lance (5) disposed below the collection matrix and within the carrier or backing material. The lance may be guided by a channel (4) in the collection matrix, so that when the device is pressed between the thumb and a finger, the lance will be forced through the channel and into the finger, thus piercing the finger and enabling a sample of blood to be collected onto the collecting matrix. Once the sample has been taken, the cover or sheath can be slid over the collecting matrix, thus protecting the sample as well as individuals handling the used device.

FIG. 4 is an enlargement of a section of FIGS. 2 and 3, showing in more detail the preferred arrangement of the lance, collection matrix and the guiding channel.

Typically, a collection device contemplated herein, in a particular preferred configuration, will have dimensions of approximately 40×20 mm and will be about 2 mm thick. However, larger or smaller collection devices may be useful in different applications and can be designed along equivalent parameters.

The collection device is primarily designed for the collection of blood and other body fluids prior to analysis of the trace element content. However, similar design principles can be used for sample collection of other fluids, omitting the integral lance. Of course, even for blood sample collection, the device described above may be provided with a separate lance, packaged together in a kit of separate components if desired.

The design of the sample collection device provides for low manufacturing costs, a robust configuration, ease of transportation, ease of storage, and can be used to collect a drop of test sample from a remote site by an inexperienced collector.

The matrix, which forms an integral part of the device, is typically an inert material with respect to fluid interaction prior to analysis and does not interfere with the subsequent sample analysis. The sample adsorbed onto or into the matrix can be stored indefinitely, without the addition of preservatives that may add contaminants to the sample.

The preferred material suitable for the matrix is cellulose, either granular or fibrous and may be either formed or preformed. Typically, the sample of blood transferred to the blood collection device does not have a specific volume. Hence the matrix may be encoded with an internal standard to normalize the analytical data on analysis.

The matrix may also be composed of inorganic materials suitable for a matrix of the ceramic-type, for example compounds of lithium, boron, carbon, magnesium, aluminium and silicon. Although this list is not exhaustive, it does encompass the main ingredients for an appropriate robust thermo-ceramic.

Typically, a sample of blood is transferred to the collection device that has a small lance or puncturing needle incorporated into the matrix, or into the backing/support material. The patient grips the device and causes a small pinprick to be administered. The collected blood does not have to have a specific volume as the matrix can be encoded with an internal standard, which normalizes the analytical date on analysis.

The device can have a laser-scannable bar code for recognition of the patient or to include any other additional information on the sample and its source. The amount of blood required is usually less than 50 μL. The device can also have a sealing mechanism to ensure that the device plus sample can be transported and will not be contaminated.

The matrix may be affixed to, or encapsulated within, the support material or holder by any known means and may employ adhesives. Further, an antibiotic barrier may be applied to prevent contamination of the sample or the analytical equipment and personnel.

The present invention also makes use of collection devices which do not possess a collection matrix affixed thereto. The collection matrix may be simply omitted and the sample applied directly to the support material (backing). This may be particularly useful in certain body fluid collection devices. In such devices it may be advantageous to introduce indentations (wells) into the support material, to allow for sample immobilization or the application of multiple samples and/or standards to the same support material (device) by application to multiple indentations (wells) in the support material.

Sample of fluids applied to any of the collection devices describe herein may be dried before analysis.

### Example 3

### Sample Analysis System

Traditionally, quantitation in LA-ICP-MS has been approached by controlling the power coupling the laser to the sample, to ensure uniform ablation characteristics and transfer of uniform amounts of solid to the analytical plasma. While this has much to recommend it when the nature of the matrix can be assured (eg. glass or similar), there are significant problems associated with standardisation of the coupling and transfer efficiency when matrices are not uniform. Furthermore, when the surface characteristics of the sample also vary it is extremely difficult to ensure uniform ablation.

Until the present invention laser ablation ICP-MS technology has been at best a semi-quantitative technique and more usually a comparative technique for the determination of trace element levels in any solid material. In this embodiment of the invention quantitation in LA-ICP-MS has been approached by quantitation of the amount of debris (ablated or ionised material) that is actually transported from the laser cell to the analytical plasma.

When using an infrared laser, where the particle size of ablated material is relatively large, Ultra-violet spectral interference can be used to quantify the amount of particles (ablation efficiency) entering the plasma. However, in the majority of cases the techniques currently employ either UV or Excimer lasers. These lasers produce particles that are too small to have sensible UV scattering and consequently relatively inexpensive particle quantitation is not possible. However, laser interferometry can be used, as an appropriate alternative technique, to quantitate the amount of ablated material and thus the efficiency of UV lasers. Once transport efficiency is quantified, it is then possible to quantify the amount of particles that are entering the analytical plasma and hence quantify the resulting signal (ie. amount of any one element).

The quantification process can be further enhanced by using internal standards in the support matrix of the collection/transportation device described above, or by adding one or more standards to the sample to be analysed. A suitable internal standard can be selected from elements which are not commonly present or are below detectable levels in a particular sample. Thus, for blood samples, elements such as Hf, Ir, Ru, Rh, Ta and heavy rare earths can be used as internal standards, and incorporated into the inert matrix by bonding to the surface of the particles used to produce the matrix, or may even be present as a natural constituent of the sample itself.

In case where the internal standard is incorporated into the matrix, when the sample is ablated, the particles of the matrix are carried into the analytical plasma along with the sample. Quantitation of the transport efficiency of all debris is achieved using laser interferometry, or an appropriate alternative technique, and supported by normalisation to the signal from internal standards. Since the bonding characteristics of the internal standards and the efficiency of absorption of the matrix are known, as is the transport efficiency, it is possible to calculate the concentration of the element in the sample adsorbed onto the matrix, in this case blood.

In another embodiment of the present invention, quantitation by LA-ICP-MS has been approached by quantitation against matrix-matched standards.

Quantitation is achieved by using internal standards in the collection matrix, or by adding one or more standards to the sample to be analysed. A suitable internal standard can be selected from elements that are not commonly present or are below detectable levels in a particular sample. Thus, for blood samples, internal standards are incorporated into the inert matrix through solution doping, or may even be present as a natural constituent of the matrix itself. The collection matrix is doped with the relevant standards to act as mass calibration standards. These may be Be, In and Bi, or other suitable combination depending upon the analysis required. In addition any other analyte can be spiked into the matrix pad and the pads analyzed. The spiking of calibration standards onto the matrix pad allows for its analysis as a “blank”. To the standard-spiked matrix pads, blood, sweat, urine or any other fluid sample may subsequently be added. The sample is dried at 105° C. for 2 hours, but may be any other suitable temperature and time, and then ablated. The sample plus the ‘under’ matrix is ablated and carried into the plasma simultaneously. Ionization is achieved for both components and, in this way samples are calibrated. Hence, because of this, the nature of the sample is not important as the sample and the matrix containing the internal standards are introduced simultaneously to the plasma. This protocol removes the necessity for a spike as the spike is already in the matrix pad on which the sample is collected. Therefore, it does not matter what the sample is, as it will be introduced into the plasma with the standards thereby overcoming any matrix interference. In this embodiment, it is not necessary to add a range of analytes to the matrix because the Be, In and Bi act as the calibrants and can be calibrated against all other elements with respect to mass response before the samples are analyzed. Of course there are a series of matrices that are spiked (detailed in text already) with standards from which calibration curves may be established thereby facilitating quantification of trace elements contained in the blood or other fluid.

Thus, fibrous cellulose matrix pads are prepared and doped with the set of mass calibration elements and dried. Blood, or other fluid is added, dried and ablated using a 10×10 matrix raster. The data are collected and read against results obtained from a concentration range (100, 200, 500 ppb etc) of multi-element standards prepared and measured in the same way. Quantitation for any matrix may thus be achieved because the standard and sample are being introduced in the same way which therefore negates potential matrix problems. The date are cross-referenced to Be, In and Bi in the standards and in the matrix with sample, and their relative values in each normalized.

The core components of the Sample Analysis System of this embodiment comprise a laser for producing an aerosol of the sample (Laser Ablation), an argon plasma, or ‘electrical flame’, operating at temperatures in excess of 7,000° C. (Inductively Coupled Plasma) in which the aerosol is ionized, a mass filter (Mass Spectrometer) for separating the ions into ‘packets’ according to their mass to charge ratio, and an ion detector (Multi-channel Analyzer or Ion Multiplier) for detecting the ions in each ‘packet’. The system operates with a routine sensitivity capable of achieving parts per billion detection limits. All data can be electronically stored for future reference.

Suitable ICP-MS system utilizes a quadrupole mass filter, controlled by alternating RF and DC fields in the quadrupole, to allow transmission of ions of one selected mass to charge ratio at any specific time. Cycling of the quadrupole allows passage of any selected ion with a mass to charge ratio of >250 amu at specific times during the cycling program. Each naturally occurring element has a unique and simple pattern of nearly integer mass to charge ratio, corresponding to its stable isotopes, thereby facilitating identification of the elemental composition of the sample being analyzed. The number of registered element ions from a specific sample is proportional to the concentration of the element isotope in the sample.

For multi-element analysis, the quadrupole is generally configured to scan at 1 Hz (once per second). Under this circumstance, if, for example, 100 isotopic masses are being analyzed, each isotopic mass will be collected only one hundredth of the entire scan time.

It will be understood that other configurations and types of instrumentation can be used with the devices and methods of the present invention without undue modification of protocols presented herein.

In one exemplary operation, the sample is introduced into a laser ablation cell and ablated, using either an Excimer or Frequency Quadrupled Nd-YAG laser, for a period typically not exceeding 30 seconds. Debris from the ablated sample passes down an interface tube, made from Nalgene as a suitable plastic material but other material could also be used, attached to the torch of an inductively coupled plasma (ICP). The sample debris passes through a zone in this tube, adjacent to the torch, into which independent laser radiation is being passed. A concentric series of dynode detectors measures the photon flux, reflected from the sample debris particles, which facilitates quantitation of particle scattering. Knowing the amount of scattering allows linear correlation to the amount of particles doing the scattering. The Laser scattering device is calibrated using conventional smoke cells.

The level of scattering is a quantitative indication of the amount of debris passing down the tube. This debris contains the sample material (blood) in addition to particles of a pre-coded (with internal standard) carrier matrix. The particles now pass on into the Inductively Coupled Plasma (ICP) where they are ionised and separated using Time of Flight (ToF) segregation. The elemental composition for the sample is established and quantified with reference to the signal obtained form each of the analyte isotopes. Quantitation of the concentration of elements present in the sample and hence the blood, is calculated with reference to the scattering signal from the Laser Interferometer. The amount of sample being analysed is normalized to the signal generation by ionisation of the components in the pre-coded matrix. In this way the amount of material ablated is used to obtain the mass component of the transported material and the elemental signature of the pre-coded matrix facilitates normalization of the response with reference to an ionisation efficiency cross comparison.

Quantitation of elements in the sample may also be achieved by incorporating standards into the sample or into/onto the collection matrix/support, or both. The pre-coded collection matrix may contain a cocktail of elements that are not naturally present in the sample such as blood or other fluid, at levels above the detection limit of the technique. These elements typically include one or more (ie. mixture of) Beryllium, Scandium, Zirconium, Niobium, Rhodium, Ruthenium, Indium, Hafnium, Tantalum, Rhenium, Osmium and Iridium. This requires doping of appropriate analytes at levels between 1 and 10,000 ng/mL to the matrix or support. The elements are chosen to cover both mass and ionisation potential ranges present in the analytically significant analytes.

In another exemplary operation, the sample is introduced into a laser ablation cell and ablated, using a Frequency Quadrupled Nd-YAG laser operating at 266 nm, for a pre-determined time interval typically dictated by the number of analytes being aquired. Debris from the ablated sample passes down an interface tube, made from Nalgene or suitable other plastic, attached to the torch of an inductively coupled plasma (ICP). The pre-coded matrix may contain a cocktail of elements that are not naturally present in blood, at levels above the detection limit of the technique. These elements typically include one or more (ie. mixture of) Beryllium, Scandium, Zirconium, Niobium, Rhodium, Ruthenium, Indium, Hafnium, Tantalum, Rhenium, Osmium and Iridium. This requires doping of appropriate analytes at levels between 1 and 10,000 ng/mL to the matrix. The elements are chosen to cover both mass and ionisation potential ranges present in the analytically significant analytes.

Readout from the spectrometer, for reporting purposes, is expressed in concentration units appropriate to clinically accepted protocols. In addition, the readout contains information on the acceptable ranges of analytes in normal healthy individuals and indicate whether the sample under investigation is below, above are in the accepted range.

The methods and devices of the present invention enable the mass screening of a variety of blood or other body fluid samples for a wide range of essential and toxic trace elements, or of samples of other fluids such as water or lubricants, for contaminants indicative of pollution or wear. Only a small volume of sample liquid (one or two drops) is required for multiple element analysis. Sample collection of body fluids does not require the use of a hypodermic needle and consequently is essentially non-invasive and considerably safer than existing methods. The sample is collected and stored in an inert matrix without need for addition of preservatives. The sample can be handled and transported safely and easily. The preferred method of analysis, quadrupole Laser Ablation-Inductively Coupled Plasma-Mass Spectrometry, is very sensitive and can detect and measure trace/ultra trace amounts of an element. The methods described herein are suited to full automation and high throughput screening and analysis of samples. Further, the methods and devices of the present invention enable multi-element testing at a significantly lower cost than many current single element tests, thus making the economical mass-screening of target populations possible.

Examples of suitable internal standards which may be used for quantitation of elements, in conjunction with the devices and methods of the present invention, are detailed in Table 1 below.

TABLE 1

| | SARM 1 | SARM 3 | SAMR 46 | |
|---|---|---|---|---|
| Sample Name | NIM-G | NIM-L | S-14 | SY-2 |
| Alt. Name | Granite | Lujavrite | Stream Sediment | Syenite Rock |
| Sample Type | ppm | ppm | ppm | ppm |
| Si | 353848 | 244936 | | 280975 |
| Ti | | 2878 | | 899 |
| Ai | 63933 | 72190 | | 63722 |
| Fe 3+ | 4197 | 61410 | | 16988 |
| Fe 2+ | 10105 | 8784 | | 27672 |
| Mn | 155 | 5983 | | 2478 |
| Mg | 362 | 1689 | | 16222 |
| Ca | 5575 | 23013 | | 56889 |
| Na | 24928 | 62093 | | 31974 |
| K | 4142 | 45741 | | 36942 |
| P | 44 | 262 | | 1877 |
| Ag | | | | 0.029 |
| As | 19.3 | 1.92 | | 17.3 |
| Au | 0.0011 | 0.00084 | | 0.00052 |
| B | | | | 88 |
| Ba | 120 | 450 | | 460 |
| Be | 7.75 | 29.5 | | 22 |
| Bi | 0.276 | 0.466 | | 0.111 |
| Br | | | | |
| Cd | 0.113 | 0.91 | | 0.21 |
| Ce | 195 | 240 | | 175 |
| Ci | 263 | 1200 | | 140 |
| Co | 0.36 | 2.44 | 54 | 8.6 |
| Cs | 1.06 | 2.78 | | 2.4 |
| Cu | 12 | 13 | 563 | 5.2 |
| Dy | 17 | 3.1 | | 18 |
| Er | 10.5 | 2.6 | | 12.4 |
| Eu | 0.35 | 1.2 | | 2.42 |
| F | 4200 | 4400 | | 5030 |
| Ga | 27 | 54 | | 29 |
| Gd | 14 | 3.6 | | 17 |
| Ge | | 0.89 | | 1.3 |
| Hf | 12.4 | 231 | | 7.7 |
| Hg | 0.0189 | 0.0445 | | 0.0043 |
| Ho | 3.6 | 0.9 | | 3.8 |
| I | | | | |
| In | | | | |
| Ir | 0.0005 | | | 0.0005 |
| La | 109 | 250 | | 75 |
| Li | 12 | 48 | | 95 |
| Lu | 2 | 0.4 | | 2.7 |
| Mo | 2.84 | 1.21 | | 0.53 |
| N | | | | |
| Nb | 53 | 980 | 28 | 29 |
| Nd | 72 | 48 | | 73 |
| Ni | 8 | 2.2 | 122 | 10 |
| Os | | | | |
| Pb | 40 | 43 | 14000 | 85 |
| Pd | 0.007 | | | 0.015 |
| Pr | 19.5 | 18.4 | | 18.8 |
| Pt | | | | |
| Ra | | | | 3.7 |
| Rb | 325 | 190 | 18 | 217 |
| Re | | | | |
| Rh | | | | |
| Ru | 0.01 | | | 0.002 |
| S | | 650 | | 160 |
| Sb | 1.19 | 0.13 | | 0.26 |
| Sc | 0.9 | 0.5 | | 7 |
| Se | 0.012 | 0.014 | | 20 |
| Sm | 15.6 | 5 | | 16.1 |
| Sn | 3.3 | 7.4 | | 5.7 |
| Sr | 10 | 4600 | 28 | 271 |
| Ta | 4.9 | 25.2 | | 2.01 |
| Tb | 3 | 0.7 | | 2.5 |
| Te | 0.007 | 0.009 | | 0.002 |
| Th | 51 | 66 | | 379 |
| Ti | 0.93 | 0.326 | | 1.5 |
| Tm | 2 | | | 2.1 |
| U | 15 | 14 | | 284 |
| V | 2 | 81 | 195 | 50 |
| W | 1.45 | 8.28 | | 0.76 |
| Y | 143 | 22 | | 128 |
| Yb | 14.2 | 3 | | 17 |
| Zn | 50 | 395 | 6200 | 248 |
| Zr | 300 | 11000 | 95 | 280 |

The collection matrix, if one is used, may be impregnated with a trace metal cocktail, of known concentration using purpose prepared aqueous solution standards. In certain preferred embodiments, the matrix may contain 2 ppm of Be, In, Hf as internal standards to calibrate the mass response for the system in blood analysis. In other embodiments describing wear metal analysis of oil, 2 ppm of Be, In and Th may be used. In yet other embodiments, different suites of elements may be used.

Separate standard matrix pads may be used to calibrate the sensitivity and these may be as follows for blood and body fluids; a single pad containing, but not restricted to, Li, Na, Mg, Al, P, K, Ca, Ti, V, Cr, Mn, Fe, Co, Ni, Cu, Zn, Ga, As, Se, Rb, Sr, Mo, Cd, Sn, Sb, Te, Ba, La, Ce, Eu, Dy, Yb, Hg, Tl, Pb, Bi, Th and U at 1 ppb, a second pad with all these at 2 ppb. A third pad with all of these at 5 ppb a fourth pad with all of these at 10 ppb a fifth pad with all of these at 20 ppb a sixth pad with all of these at 50 ppb a seventh pad with all of these at 100 ppb an eight pad with all of these at 200 ppb a ninth pad with all of these at 500 ppb a tenth pad with all of these at 100 ppb. An appropriate concentration can then be used for the set of elements being determined in a particular fluid sample. In another embodiment, a suite of elements appropriate to wear metal analysis in oil, for example, Li, B, Mg, Al, Si, P, Ca, Tl, V, Cr, Mn, Fe, Co, Ni, Cu, Zn, Ga, As, Se, Sr, Y, Zr, Mo, Ag, Cd, Sn, Sb, Ba, La, Ce, Hf, Hg, Pb and U may be doped into matrix pads at 1 ppb through 1000 ppb as above, so that when ablated, a range of elements across the mass spectrum may be used as internal standards to standardize the system. Thus, the collection matrix, when used, may contain a pre-calibrated concentration of selected analytes. Both a broad-spectrum general collection matrix/device and a test specific matrices/device/s may be employed for specific elements or suites of elements. Further, any one, or combination or range of internal standards analytes may be spiked into the collection device to ensure its broad spectrum or specific use. For example, for broad spectrum, the preferred combination is, Li, Na, Mg, Al, P, K, Ca, Ti, V, Cr, Mn, Fe, Co, Ni, Cu, Zn, Ga, As, Se, Rb, Sr, Mo, Cd, Sn, Sb, Te, Ba, La, Ce, Eu, Dy, Yb, Hg, Tl, Pb, Bi, Th and U and for specific applications, for example analyzing oils preferred is, Li, B, Mg, Al, Si, P, Ca, Ti, V, Cr, Mn, Fe, Co, Ni, Cu, Zn, Ga, As, Se, Sr, Y, Zr, Mo, Ag, Cd, Sn, Sb, Ba, La, Ce, Hf, Hg, Pb and U and for blood the preferred combination is, Li, Na, Mg, Al, P, K, Ca, Ti, V, Cr, Mn, Fe, Co, Ni, Cu, Zn, Ga, As, Se, Rb, Sr, Mo, Cd, Sn, Sb, Te, Ba, La, Ce, Eu, Dy, Yb, Hg, Tl, Pb, Bi, Th and U.

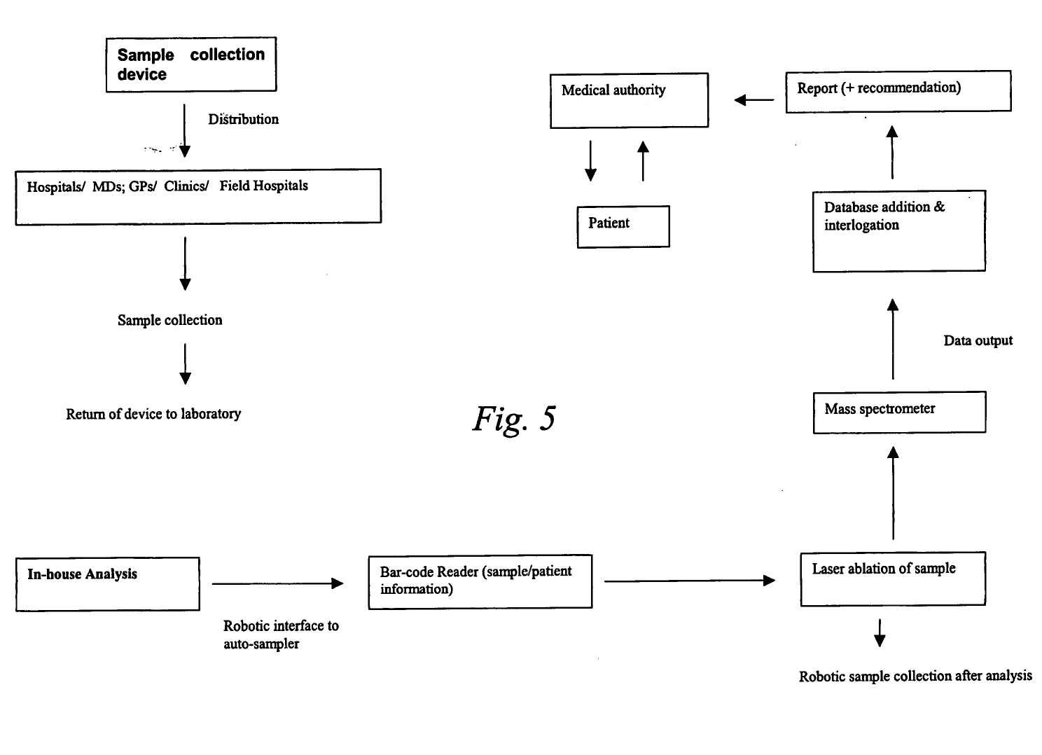

A typical procedure of collecting and analyzing a sample is summarized in FIG. 5. Of course, manual procedures can also me adopted, as can variations of the proposed exemplary scheme.

### Example 4

### Analysis of Collection Matrices

The purpose of the experiments described below was the definition and/or refinement of chemically and mechanically robust fluid adsorption/absorption matrix/matrices to facilitate the collection and quantitative analysis of micro-litre fluid samples by Laser Ablation-Inductively Coupled Plasma Mass Spectrometry (LA-ICP-MS). For purposes of this example fluids under consideration are blood, urine and oil. However it will be understood that any other fluid, biological or otherwise, may be analysed using similar matrices and techniques.

Preferably the sample collection matrices should be suitable for incorporation into a robust, transportable sample collection device. The device should have specific attributes such as but not limited to:
be cheap and capable of precision mass production;
be small and easily accommodated in laser cells for ablation prior to analysis;
be able to be coded for automatic pre-analysis reading and referral of the sample back to the data, and the data to the client;
for blood collection, contain a mechanism for penetration of individual patient's skin thereby minimising potential ‘stick injuries’. There would be some form of shielding device, or mechanism, that would “shield” the puncturing mechanism such that it would not be able to penetrate the skin of another person subsequent to initial collection of blood;
produce minimum biohazard with material after analysis and prior to disposal. This implies a small collection device and a small blood sample (less than 100 μL), and a very small amount of material comprising the sampling device itself that would ultimately have to be incinerated;
easy transportability to and from the collection site and through conventional mailing procedures. The device should be such that conventional postal systems can be used without the possibility of contamination and release of potentially bio-hazardous material; and
be capable of being used by non-medical personnel.
   Matrix Materials

The original preferred matrix material used for process testing was fibrous cellulose. Using this material, it was possible to readily form backed cardboard ‘punch-outs’ containing the cellulose absorptive medium. Micro-litre samples of blood, added to this material, were qualitatively analysed by LA-ICP-MS. Qualitative spectra and raw count data were generated, much of which reflected trace metals in the absorbed blood. However, it was reasoned that the cellulose, being a natural organic product, might be contributing to the analyte signal of a range of elements recorded. Hence, it was determined that cellulose, together with an array of other potential matrix materials, be further investigated, both in terms of its chemical and physical characteristics.

Some attributes of suitable sample collection matrices include but are not limited to:
must be chemically “clean”, that is, have a low concentration of analytes of interest;
robust, that is, capable of transportation, often over long distances without fragmentation;
have significant wettability, both by aqueous and non-aqueous (blood and oil) samples while still retaining integrity;
capable of withstanding laser ablation removal of samples; and
not contribute to analyte segregation during analysis.
   Matrix Choice

The parameters detailed above govern the choice of matrix and, as such, preclude certain materials. A list of matrices investigated follows with indications as to their potential suitability, or otherwise, which resulted in a final short list of potentially useful material to be subsequently tested. The choice of white metal oxides as potential matrices is based on the fact that the two detailed herein are locally manufactured in bulk, are extremely cheap and, using the modern generation of UV lasers (unlike IR lasers), are customarily considered not to have variable coupling efficiencies between light and dark matrices.

Potential organic and inorganic matrix materials investigated are:
Pig-toe mussel shell (aragonite)—sourced from the WA pearl industry
Aluminium hydroxide—Alcoa (WA)
Titania—New Millenium (WA)
Bacterial grade glucose—sourced by Professor Watling
Starch “A”—BDH Analar analytical reagent
Starch “B”—Ajax Chemicals Univar analytical reagent
Glucodin—Boots Healthcare Australia
Cellulose—high purity powder—Sigma Chemicals Microgranular
Cellulose—high purity fibrous cellulose_Sigma Chemicals Medium Fibrous
Hydroxy Butyl Methyl Cellulose—Sigma Chemicals
Flour—rice, maize, wheat, soy, rye and corn flour commercially available grocery lines

All of the above matrices can be used for lubricants where the levels of metals are much higher. However, the following are particularly useful choices of matrices for blood and other body fluid analysis, which can also be used for analysis of lubricants or water samples.

Aluminium hydroxide [Al(OH)₃]: A very high quality aluminium hydroxide is produced in Western Australia. It is analytically relatively clean and cheap, and is being considered as a matrix.

Cellulose: Cellulose is an excellent theoretical matrix choice in that it is typically low in heavy metal concentration. A variety of ultra-pure cellulose was tested for compactability, wettability and metal content. The physical characteristics of cellulose as such (it was the original matrix) make it important material as a potential matrix. Particularly useful is fibrous cellulose in the form of—cellulose filter papers (Whatman 540, but also 541, 542 and other cellulose filter papers, Whatman International Ltd, Maidstone, England).

Flour: Newly acquired rice flour has proved exceptionally robust under wetting and drying conditions and may also be advantageously used as a matrix.

In addition to simply using the matrix material as supplied, relevant matrices were leached and the leached residue tested to see if significant metals could be leached, thereby reducing the metal content of the matrix and possibly rendering it more useful by lowering the level of contaminant metals, or actually reducing the level of metals in the sample to a level where previously unsuitable material would now be suitable.

### EXPERIMENTAL

(I) Chemical Characterisation

Solution ICP-MS: In order to assess the ‘purity’ of the respective potential matrices, appropriate sub-samples of water-soluble materials were dissolved in Milli-Q (mQ) water and made to volume. Water-insoluble samples, (primarily the inorganic materials) were subjected to both cold and/or hot (or both) hydrochloric, nitric, aqua regia and nitric-hydrofluoric acid leaches. The leachates were recovered, made to volume, appropriately diluted and analysed by solution introduction ICP-MS. The leached residues were recovered and a selection of sub-samples subjected to total dissolution followed by solution ICP-MS analysis using a VG PlasmaQuad 3 ICP-MS made by VG Elemental, Ion Path Road 3, Winsford, Cheshire CW7 3BX, United Kingdom. Further selected residue sub-samples, along with unleached equivalents, were subjected to total acid dissolution, made to volume, diluted and again analysed by solution introduction ICP-MS.

The solution experiments facilitated elimination of several of the potential matrix candidates, having unacceptable concentrations of analytes of interest in the raw material and analytes little, or not adequately, reduced by acid leaching. The ‘solution’ assessment indicated that cellulose and aluminium hydroxide were the best candidates but that both of these may contain certain analytes of interest. Because of the need to dilute the solutions for ICP-MS analysis, very low apparent concentrations in solution frequently translated to significant concentrations in the sample when corrected for mass and dilution; In many cases, these analytes may not be present or, if present, present at very much lower concentrations. To test this thesis, ‘raw’ sub-samples, and corresponding leached residues where applicable, were pressed into ‘briquettes’ (see below) and subjected to comparative qualitative UV LA-ICP-MS analysis.

Laser Ablation ICP-MS: It is not necessary that the sample matrix will contribute an equivalent amount of material to the analytical sample as the blood or other fluid. The incorporation of the matrix and its ionisation will not be equal to that for the blood contained in it. Because of this, the contribution of matrix to the analytical signal will not necessarily be in proportion to its relative matrix/blood ratio. Hence, it was necessary to determine what relevant contribution the matrix has to the analytical signal during a real analysis. Laser ablation analysis of the matrix was therefore also undertaken. Because the use of argon as a carrier gas is the traditional method of transport of ablation debris to the plasma this was the initial gas used for all experimental purposes. However, helium is finding an increased following in the scientific community as a transport gas as it often gives improved sensitivity and reduced isobaric interferences. Consequently this gas was also investigated.

(II) Physical Characterisation

Physical characterisation of potential matrix materials included assessment of compaction integrity, both at 500 and 1000 kg/sq in, wettability to blood and aqueous solutions, integrity after sample addition, contrasting behaviour of single and multi-component matrices, and internal standard introduction. Results from some of these investigations are detailed below.

The use of an internal standard is necessitated because of the variability in ablation efficiency between samples. There is no way of controlling the “fluence” variation (variation in the efficiency of coupling and hence power transfer of the laser energy to the sample) from sample to sample. Because of this, varying amounts of analyte will reach the plasma depending on the relative fluence between samples. Consequently, it is necessary to ensure that there is a mechanism for estimating the amount of material being transported to the plasma for each sample. The method used for an infrared laser was to measure the scattering of light by the transported particles. However, this mechanism is not possible when a UV laser is used (the laser used for these experiments was a frequency quadrupled Nd-YAG UV Microprobe Laser System operating at 266 nm in pulsed Q-switched mode. The Laser System was manufactured by VG Elemental, Cheshire, United Kingdom.

However, spiking a simple element cocktail into the matrix, either prior to, or concurrent with, sampling provides a useful and inexpensive internal standard for quantification experiments.

Results and Discussions

Details of eighteen experiments completed during the period October-December 2002 are set out below. Sixteen of the experiments relate specifically to physical and chemical characteristics of the matrix, and analysis of absorbed aqueous standard, mineral CRM and blood samples. The remaining two experiments, Experiments 13 and 15, deal with the analysis of oil samples—these are reported together at the end of this section.

The resulting analytical data is presented in a series of Appendices identified by experiment number, for example, ‘Appendix Experiment 12’. These appendices should be viewed in conjunction with the relevant commentary on the individual experiments as contained herein. Frequently, averages of data and % standard deviations (coefficient of variations) have been computed.

In most appendices, isotopic data has been computed to 100 percent elemental concentration using natural isotopic abundance relations. In a small number of cases, data is presented solely as isotopic concentrations at the measured isotopic mass. This is clearly indicated in the respective appendices.

In an attempt to optimise signal response, peak hopping instead of normal scanning acquisition was employed. Under this analytical regime, data acquisition at each isotopic mass occurred on three channels only. Not uncommonly, transient electronic spikes may be recorded on one of the three channels. The on-board computer processes the data from all three channels and reports the results as raw count ‘concentrations’. Where a measurement includes a transient spike, the resulting raw counts for that analyte may be considerably elevated relative to duplicate or replicate analyses of the equivalent analyte in the same sample. This leads to often-marked concentration contrasts for specific analytes in these samples. The problem may be overcome by increasing, to say seven, the number of channels over which individual isotopic mass data is collected. Under these circumstances, a normal ‘smoothing’ algorithm may be automatically applied across the seven channels to produce precision results for duplicate or replicate analyses. Having established this as being a major cause of analyte variability, analytical protocols have been appropriately modified to allow data collection over the increased number of channels.

Another cause of analyte variability may be due to possible surface ‘contamination’ of the collection matrices. To minimise contamination, the top pad of a matrix wad has been removed so that there is no airborne contamination on the surface to be analysed. In an embodiment of this process, the matrix pads are prepared in a sterile, dust-free clean room, enclosed in a container which may only be breached immediately prior to sample collection. Improved analytical precisions, following implementation of this protocol, are attributed to the sample preparation

Correction of data for identified transient spikes had led to a marked improvement in analyte reproducibility and, hence, ‘precision’ data.

### Example 5

### Matrix and Blood-Related Experiments

Experiment 1

The aim of this experiment was to develop and test procedures to produce 3 mm diameter test tablets as a prelude to physical characterisation of sample matrices. For this purpose, an XRF pressed powder vacuum press was modified, and new dies manufactured, to facilitate pellet production. Matrix materials chosen for the inaugural production tests were glucose, cellulose and a 1:1 mixture of the two; initial compaction pressure was 500 kg/sq in. Initial physical and chemical investigations were undertaken concurrently until preferred matrices were identified.

Pelletising of glucose required the use of weighing paper between sample and metal on the press die. Absorption of liquid appears good.

Cellulose pelletised quite well, with very good strength. However, fluid absorption was slow. A 1:1 mixture of glucose and cellulose powder pelletised well without the need for weighing paper between pellet and die. Pellet strength was improved over glucose alone and fluid absorption was intermediate between rates for glucose and cellulose powder pellets compacted at equivalent pressure.

Experiment 2

The principal objective in this experiment was to assess the chemical purity of a range of potential matrix materials. Sample preparation for analysis was undertaken concurrently with pelletising press modifications. Various matrices, including pig-toe mussel shell, glucodin, glucose, cellulose, hydroxy butyl methyl cellulose (HBM cellulose), TiO₂ and Al(OH)₃ were leached, dissolved or digested in preparation for solution ICP-MS purity assessment.

Method

Pig toe mussel (Sample A, B, C and D)—˜1.5 g pearl seed taken, dissolved in 20 mL 1:1 HCl:mQ water, then taken to dryness. 4 mL of HNO3:mQ 1:1 added, heated and made up to 100 mL with mQ water. Diluted ×20 with mQ (2 ppb Ir, Rh) water for ICP-MS.

Glucodin (Sample E and F)+Glucose (Sample G)—˜1.5 g Dissolved in 100 mL of mQ water. Diluted ×5 for ICP-MS.

Cellulose (Sample H)+HBM Cellulose (Sample I)—˜0.5 g digested in 20 mL cHNO3 for 36 hours, reduced to 10 mL and made up to 100 mL with mQ water. Diluted ×5 for ICP-MS.

TiO₂ (Sample 001)+Al(OH)₃ (Sample 003)—Leached with 1:1 HCl:mQ water for 36 hours, decanted and washed 3 times with mQ water (˜20 mL). Decanted solution (leachate) made up to 100 mL with mQ water. Diluted ×10 for ICP-MS.

TiO₂ (Sample 002)+Al(OH)₃ (Sample 004)—Leached with 1:1 HNO3:mQ water for 36 hours, decanted and washed 3 times with mQ water (˜20 mL). Decanted solution (leachate) made up to 100 mL with mQ water. Diluted ×10 for ICP-MS.

Residues were dried and saved for LA-ICP-MS.

This experiment was concerned with the determination of the trace element concentrations in prospective matrices for blood (and other fluid) collection, together with looking at some of the results of leachates of titanium dioxide and aluminium hydroxide.

The results for the leachates are detailed (Appendix Experiment 2). It may be possible to indicate that aluminium is obviously leached from the aluminium hydroxide matrix, but also from the titanium dioxide matrix, and conversely titanium is leached from the titanium dioxide matrix and there is also some indication of leaching of titanium from the aluminium hydroxide matrix. In the case of titanium dioxide, HCl appears to be more aggressive than HNO₃, whereas the reverse is the case for the aluminium hydroxide. Concentrations of manganese, copper, strontium, zirconium are found from the leachates of both matrices while zinc, rubidium, barium and lead appear to be quite concentrated in leachates from the titanium dioxide matrix. In the aluminium hydroxide matrix tin, gallium, zirconium, hafnium and uranium appear to be present in leachates from this matrix.

Total digest and/or solubilization data of pig-toe mussel, glucodin, glucose, cellulose and HBM cellulose are also presented in Appendix Experiment 2. The pig-toe mussel contains significant concentrations of lithium, aluminium, titanium, manganese, copper, zinc, rubidium, strontium and barium. While this would imply that the matrix is not suitable as a blood collection matrix, because of the concentration of these elements, it is also necessary to analyse the pig-toe mussel material with sample attached under laser ablation conditions rather than solution conditions to make sure that these elements are also carried over by laser ablation and not just present in total digests. In the case of glucodin, glucose, cellulose and HBM cellulose all contain significant amounts of aluminium, titanium, chromium, manganese, nickel, copper, zinc, rubidium, strontium and barium while cellulose matrix alone, in addition to containing these elements, also contains significant concentrations of lead and bismuth; both cellulose and HBM cellulose also contain concentrations of zirconium, tin, thallium and thorium not found in the glucodin and glucose.

Although these matrices all contain significant amounts of trace elements in the ppb range, this does not necessarily preclude them from use as a sample collection matrix as conventional blank correction can be used to overcome problems associated with blank content. This can be further emphasised by the fact that inter-element ratios could be used to determine, and to augment, blank corrections by looking at relationships between metals and tracing these through to the final analytical protocols

Experiment 3

The purpose of this experiment was to further test, the pelletising and adsorption characteristics of cellulose powder, glucose, and starch, and mixtures thereof, and to check the dissolution/absorption characteristics of the pellets by SY-2 (mineral CRM, Canadian Certified Reference Material Project (CCRMP), Table 1 solution. The results of Experiment 3 are set out in Appendix Experiment 3

Cellulose powder alone works well. The glucose undergoes surface dissolution leaving holes on the surface. The starch absorbed water and expanded, causing the surface to bulge. Under the pelletising pressure of 500 kg/sq in, the cellulose powder is tightly compressed and it takes some 10 to 15 seconds for fluid absorption. This suggests that a more fibrous cellulose with an ‘open’ structure may be preferable. To this end, further experimentation with fibrous cellulose is indicated. In addition, further experimentation with powdered cellulose at differing packing pressures is warranted.

Experiment 4

The aim of this experiment was to assess the absorptivity and mechanical stability of cellulose powder pellets compacted under differing pressures. In the first instance, powdered cellulose was suspended in mQ water and vacuum filtered. The collected filter cake was mechanically incoherent. This caused it to flake and fall apart. However the adsorption of solution was rapid.

Cellulose powder compacted under a pressure of 100 kg/sq in, while mechanically robust, still absorbed slowly. At low compaction pressure, estimated to be about 50 kg/sq in and achieved by turning the tightening screw on the press just until there was resistance, the resulting pellets illustrated rapid absorption. Furthermore, the pellet holds together well. The experiment appears to confirm that compaction destruction of porosity rises with increasing pressure thereby rendering the matrix progressively less absorptive.

Experiment 5

The aim of this experiment was to quantitated trace elements in a blood sample using internal standards. The experiment also tested the absorption of SY-2 (mineral CRM) and blood onto cellulose pellets, robustness of the doped pellets when subjected to LA-ICP-MS analysis, assess levels of possible contaminants, evaluate results arising from the doped matrices and assess the comparability between ‘wet’ and ‘dry’ matrices.

The following instrument settings were used: Lens voltages—Lens 1, 2, 3, and 4 respectively −10.8, −22.6, 0.7 and −13.3 Volts, Collector—4.6 Volts and Extraction, −332 Volts; Gas Flows—Cool gas 13.6 L/min, Aux gas 0.81 L/min Neb gas 0.74 L/min and Oxygen gas 0.00 L/min; Torch box positions—X, Y and Z axes respectively 932, 165 and 250 steps: Multiplier voltages—H.T. pulse count −2634 Volts and H.T. analogue) Volts; Miscellaneous settings—Pole bias −2.2 Volts, R.F. power 1500 Watts, Perl speed 0%; PlasmaScreen is OUT, S-Option pump is OFF.

Samples of blood were obtained from a subject with the aid of a SoftTouch lancet device (used for home blood glucose testing and manufactured by Boehringer Mannheim, Germany) applied to a pre-cleaned (absolute ethanol wiped) area of a fingertip. Successive drops of blood were encouraged to form through application of pressure. The drops were directly ‘touch’ applied to 3 mm diameter by 2 mm deep sample collection matrix tablets formed by pressing granular cellulose (Sigma Chemicals Microgranular powder) under a load of 500 kg/sq. in. The matrix tablets were affixed to a Perspex disc, 37.5 mm in diameter and 6 mm deep, fabricated from Perspex rod, using 3M Scotch Permanent Double Stick Tape. The volume of the drops was estimated to range between 30 and 70 microlitres. No preservatives or anticoagulants were used and there was no requirement to store the blood prior to application to the collection matrix, or subsequent analysis. However, there is provision for loaded sample collection matrix tablets to be refrigerated and stored following oven drying at 60° C. for one hour.

Four blood samples were prepared; two were oven dried and two were maintained “damp”. Duplicate sets of equivalent SY-2 CRM-doped (Syenite, Canadian Certified Reference Material Project) matrix pellets were prepared by pipetting 50 μL of the standard solution onto the respective matrix tablets and drying thereby generating matrix matched standards. The SY-2 CRM contains calcium, iron, magnesium, potassium and so forth and this provides a high ion flux that is possibly equivalent to the ion flux expected of blood. Hence, any ion effects that were taking place would be comparable in the blood and SY-2, as compared with a straight aqueous standard solution.

The sample holder, with affixed blood- and CRM-doped matrices was placed into the laser ablation cell of the UV Microprobe Laser System attached to a VG PlasmaQuad 3 ICP-MS both manufactured by VG Elemental, United Kingdom. The laser is a frequency quadrupled Nd-YAG operating at 266 nm; 10×10 matrix raster ablation of the samples was undertaken in pulsed Q-switched mode at a fluence of 6.2 milljoule for 60 seconds.

The output data was acquired as raw counts from on-board software and exported into Excel and manipulated. No algorithms were used for computations. The raw count date for both blood and CRM samples were matrix blank corrected by subtracting the averaged matrix blank value from the individual blood and SY-2 values. From these corrected data % Standard Deviations were computed as a measure of precision. Finally, trace element compositions for the 11 analytes examined in the exemplary run were computed with reference to matrix matched SY-2 CRM values.

Data obtained is set out in Appendices Experiment 5A and 5B.

As indicated above, part of the experimental design was to determine whether it was necessary to fully ‘dry’ the sample prior to analysis. Collection of blood onto a matrix without the drying step as detailed above, may lead to a sample being slightly damp. Hence, it was necessary to determine whether variation in the moisture content of the matrix would affect the readout of concentration of elements in the matrix. Consequently two sets of samples of cellulose were set up and, in addition to ‘wet’ and ‘dry’ blood, SY-2 certified reference material doped samples were also prepared in an attempt to quantify the concentration of metals in the blood. Blood samples and SY-2 were spiked onto cellulose in duplicate and one set of blood samples was analysed ‘wet’. A second subset was taken and dried (as above) and the samples were analysed dry. Data from these experiments is also presented in Appendix Experiment 5A

Following analysis, results for the wet samples were blank corrected and data produced. Simple inspection of the data for the ‘wet’ blood samples indicates relatively high variability in analyte to concentrations particularly in the case of lead and zinc where a variation of ±100% is recorded. The analysis of SY-2 certified reference material is far more uniform.

For the dry sample, the results are better. Reproducibility is improved and results are more uniform. From the blank corrected values for the dried blood sample it can be seen that, with the exception of barium, the results are meaningful. Barium results go negative and this is probably due to the fact that the barium signal is small relative to the blank—the blank is quite high. However, both lead and zinc are much improved and, if these are used to calculate concentrations of these elements in the blood, based on SY-2 concentrations (calculated in Appendix Experiment 5B) the blood values and expected blood values from the literature are quite close for the analytes under consideration. SY-2, a certified reference material, has been used for a number of reasons. First, use of simple aqueous solution on the collection matrix would not, on ablation, have provided a significant ion flux. The SY-2 contains calcium, iron, magnesium, potassium etc (see Table 1) and this provides a high ion flux that is possibly equivalent to the ion flux of the blood. Hence, any ion effects that were taking place would be comparable in the blood and SY-2, as compared with a straight aqueous solution. Thus a normal CRM, that has a relatively high matrix concentration will suffice.

The above experiment, including instrument settings and internal standardisation as described, is equally applicable to simpler biological fluid samples such as components of whole blood (eg. serum or plasma), urine, sweat, tears, cerebrospinal fluid and the like. The sample collection, handling and analysis of such fluids is simpler and thus greater accuracy can be achieved.

Experiment 6

This experiment was conducted to analyse the titanium dioxide and aluminium hydroxide matrices, both before and after leaching (leached residues from Experiment 2). The data produced in this experiment ties in with the leachate data from Experiment 2. Upon total dissolution, solutions derived from titanium dioxide have very high concentrations of titanium, while those derive from digestion of aluminium hydroxide are similarly rich in aluminium. Accordingly, these two elements have not been measured.

The purpose of the experiment was to evaluate the efficacy of acid cleaning of the white oxide matrices. Hence, appropriate sub-samples of ‘raw’ titanium dioxide and aluminium hydroxide, together with their hydrochloric- and nitric acid-leached equivalents, were digested in a sulphuric/hydrofluoric acid, made up to volume, diluted and analysed by solution introduction ICP-MS. The leachates derive from HCl- and HNO3-leaching of bulk titanium dioxide and aluminium hydroxide were analysed in Experiment 2 and the results reported in Appendix Experiment 2.

The comparison of the “raw” original material and the HCl- and HNO3-leached residues show that, for titanium dioxide, its HCl-leached residue and associated leachate, weak to strong leaching of lithium, manganese, copper, zinc, gallium, rubidium, strontium, (zirconium), barium, lead, (thorium) and uranium has been achieved. Here, there is generally a good mass balance between concentration in the original versus the sum of concentrations in the leachate and leached residue. In contrast, concentrations of vanadium, chromium, nickel, germanium, yttrium, zirconium, niobium, tin, antimony, hafnium, tantalum and tungsten in the raw material are unaffected by HCl-leaching.

For titanium dioxide, its HNO₃-leached residue and associated leachate, weak to strong leaching of lithium, (chromium), manganese, copper, zinc, gallium, rubidium, strontium, (zirconium), barium, lead and (thorium) is evident. In contrast, concentrations of vanadium, (chromium), nickel, germanium, yttrium, niobium, tin, antimony, hafnium, tantalum, tungsten, (thorium) and uranium are little or unaffected by HNO₃-leaching.

Turning to the aluminium hydroxide matrix, HCl and HNO₃ both have a similar leaching response with both acids weakly to strongly leaching all elements occurring in significant concentrations in the aluminium hydroxide matrix. The elements involved are lithium, beryllium, chromium, manganese, copper, gallium, strontium, zirconium, tin, hafnium, thorium and uranium. Hence, use of these acids to pre-dean the matrices is recommended. Both can be leached quite easily in both HCl and HNO₃.

Of particular importance is the presence of gallium in the aluminium hydroxide matrix. A small amount is acid-leached but this does not impact its potential of being used as an internal standard; the same holds true for zirconium. Although not as high as zirconium in the titanium dioxide matrix, zirconium in aluminium hydroxide could still be used for a double internal standard based on gallium and zirconium. There is a possible problem with the aluminium hydroxide matrix in that there is copper in it but the copper tends to be relatively uniform and if copper results in previous analyses are considered, reasonable results for copper are obtained by doing blank corrections. It should be remembered all the time that although these metals are present in the matrix, they may not contribute an equivalent amount to the determination of metals in blood because they are not transported as much as the blood to the plasma. The blood tends to fill interstices and sit on top of the matrix; hence, these elements may not contribute a significant amount to the concentrations that are present in analysed, so-called blood.

This experiment demonstrates that it is possible to variably reduce and/or eliminate a range of trace elements from titanium dioxide and aluminium hydroxide matrices. When combined with previous experiments, it would appear that possibly two matrices, aluminium hydroxide and cellulose, may constitute particularly suitable matrix materials.

Experiment 12

The purpose of this experiment was to examine the efficacy of a fibrous cellulose mat (Whatman 540 filter paper, Whatman International Ltd) as a sample collection matrix. This material is an efficient absorber of fluids, but its ‘coarse’ fibrous texture may result in variable ablation characteristics. Six duplicate sub-samples of the cellulose mat were taken and pre-prepared as follows; Two duplicate sets were rinsed for 10 minutes with 50% aqua regia and dried; a further two duplicate sets were washed overnight in aqua regia and dried while the remaining duplicate sets were left unwashed. One set each was doped with 2 ppm multi-element standard and dried whilst the second set of each was retained as blanks. It was observed that the fibrous cellulose mat, rinsed for 10 minutes with aqua regia, upon drying was rendered ‘harder’ than the other two (unwashed and overnight washed) mats.

The blanks and doped equivalents were analysed by LA-ICP-MS and the results of analysis are recorded in Appendix Experiment 12. Upon ablation, it was observed that for the ‘hardened’ rinsed matrix, the laser penetrated through the whole mat, whereas for the other two, the laser did not penetrate all the way through. This observation clearly implies that the contrasting physical characteristic of the fibrous cellulose mat impact upon laser penetration and, hence, lasing characteristics. With reference to the relevant Appendix, pages Experiment 12/3 and 12/4, it is clear that, for cerium-normalised data, date for the ‘hardened’ rinsed fibrous cellulose mat, which exhibited complete laser penetration, gives rise to the best overall precision data. Indeed, most analytes have precisions of less than 10% and frequently less than 5%. This outcome further emphasises the potential value of fibrous cellulose as a matrix material.

Experiment 16

The objective of this experiment was to evaluate potential sensitivity improvements for aqua regia and ammonium fluoride (NH₄F) doped 3:1 Al(OH)₃:cellulose matrices.

From a 3:1 Al(OH)₃:cellulose mixture, six triplicate sets of pressed pellets were prepared. These unwashed triplicate pellet sets were affixed to a Perspex disc. One set was left ‘blank’ and a further set was doped with 1 ppm multi-element standard; both were oven baked. Two of the remaining four triplicate sets were doped with 5 μL of 50% aqua regia and oven at 105° C. for 2 hours; the remaining two triplicate sets were doped with 5 μL of 1M ammonium fluoride (NH₄F) and oven baked. One set each of the aqua regia and ammonium fluoride treated pellets were further doped with 1 ppm multi-element standard and dried.

A further sample of the 3:1 Al(OH)₃:cellulose mixture was washed with aqua regia, rinsed and dried. This material is referred to as the washed matrix. From this washed matrix, equivalent triplicate sets of pellets were prepared as for the unwashed matrix described above. It was observed that the 50% aqua regia doped matrices were not as mechanically robust as other matrices prepared in this experiment. All triplicate sets were analysed by LA-ICP-MS. The results for the unwashed matrices are presented in Appendix Experiment 16A while those for the washed matrices comprise Appendix Experiment 16B.

When results for unwashed material, that is, no aqua regia wash, are considered, it is apparent that the results are significantly better for unwashed, than for the washed, material. For blank corrected matrices, normalised to cerium, precisions for the unwashed material are better than those of the washed matrix. This outcome suggests that there is no fundamental need to wash 3:1 Al(OH)₃:cellulose matrix.

Disregarding, the blank corrected, cerium normalised data for the present, and considering only the ‘raw’ 1 ppm doped matrix data, the recorded precision measurements for both unwashed and washed matrices show a general improvement in the NH₄F doped matrices. This apparent improvement in sensitivity may result from improved ablation of the matrix possibly through production of a more volatile atmosphere in the presence of NH₄F.

Experiment 18

The several previous experiments have sought to identify appropriate clean matrix materials together with preferred compaction, absorption, ablation and pre-treatment characteristics. Particularly preferred matrix and analytical conditions for most test samples, and particularly useful for blood and other body fluid samples, were identified as Whatman 540 filter paper, ablated at 10 Hz at a fluence of between 4 and 9 Milljoule with a flow of argon between 900 and 1000 mL per minute.

In the course of this work, consideration was given to the question as to whether it may be possible to prepare a blood sample in such a way that it was matrix supported, rather than matrix absorbed. If this could be achieved, then it may be possible to ablate blood samples free of matrix. In this way, analytes present in the analysis would be derived from the blood alone. Consideration of direct analysis of supported, rather than matrix-absorbed blood, arose from the observation that, during the experimental procedures segregation of blood serum and plasm appeared to occur. The observed probable segregation was not considered to be a significant problem; the laser ablation protocol was designed in such a way that the laser would penetrate through any dispersion front in the matrix, thereby sampling any segregated blood and consequently ‘re-assembling’ or re-combining the analyte cocktail. Nonetheless this observation suggested that it might be possible to overcome any potential matrix interference by ablating only dried blood.

It was reasoned that if a shallow, 3 mm diameter, 125 micron deep, depression was cast into the surface of the matrix pellet, then a drop of blood delivered to the depression would flow to fill the depression and present a flat surface away from the depression lip (meniscus) for subsequent lasing. A requirement would be that no chromatographic segregation of serum and plasma occurred. To this end, it was further reasoned that if the 3:1 Al(OH)₃:cellulose powder was compacted under high pressure (at least 1 tonne/sq in), then the matrix may be rendered effectively impervious and simply support blood as it coagulated and dried.

Consequently, a new die for the vacuum press was fabricated to produce a 6 mm diameter pellet into which was impressed a 3 mm diameter by 125 micron deep, flat bottomed circular depression. An appropriate number of new pellets were pressed at 1 tonne/sq in pressure.

Micro-litre samples of blood were delivered to, and contained within, the surface depressions on the surfaces of ten matrix pellets; five of these pellets were air dried at ambient temperature and the remaining five oven dried at 60° C. A further two blood drops were applied to the Perspex mounting disc and dried. Here, the surface of the dried blood drops was not flat, but rather, strongly undulating.

On application, it was clear that some plasma segregation and absorption occurred, causing 8 volume increase and expansion in the tightly compressed cellulose powder. However, the pellets retained sufficient mechanical integrity to allow LA-ICP-MS analysis. When ablated, the ‘serum’ tended to fragment in ‘chunks’ giving rise to somewhat variable results. Notwithstanding, the counts obtained were reasonable for most elements.

For the matrix free blood drops, dried onto the Perspex support, the ablated blood was far more coherent, with nice ablation. However, as noted above, the surface was strongly undulating leading to changed laser focal conditions and, hence, non-optimal results.

Given that the aluminium hydroxide:cellulose matrix was not impervious, the matrix free approach described above can be adopted, ie. use impervious substrate, such as Perspex, into which 3 mm diameter by 125 micron deep circular impressions have been pressed, moulded or machined. Each sample collection device can contain two such depressions, one for a matrix-matched, trace metal-doped standard reference blood, and the second to contain and confine the unknown blood sample. Alternatively, a matrix-matched, trace metal-doped reference blood could be inserted into the analytical run such that each unknown had a standard immediately adjacent to it. This would lead to 33% reference samples in the analytical run as opposed to 50% if standard and unknown were applied to the same collection device.

The results from this Experiment are presented in Appendix Experiment 18. This experiment examined heat and air-dried blood partially absorbed into an aluminium hydroxide:cellulose powder matrix, and matrix-free blood dried onto an impervious Perspex substrate.

If the corrected and normalised “no-matrix” blood is examined, the numbers are reproducible. Indeed, values are commonly comparable to the dried material. In the ‘no matrix’ blood, both mercury and lead are recorded and the reproducibility of lead is with a precision of 14%. Good numbers are also recorded for uranium on the dried material, but in the blood matrix alone, the numbers are considered to be ‘below detection limit’, consistent with a matrix uranium background and anticipated absence in the blood.

### Example 6

### Wear Metal Analysis in Oils

Experiment 13

The objective of this experiment was to carry out pilot analysis of wear metals in engine oil. It is held that the technology being investigated is equally applicable to the analysis of wear metals in oils, and that wear metals analysis is a major global industry aimed at early detection and prevention of catastrophic plant failure. Such early detection is of particular importance to the military, airline, shipping and mining industries where component failure (automotive, heavy machinery, weaponry and the like) may lead to tragic loss of life and destruction of expensive plant.

Oil from the engine of a ‘new’ Ford Fairlane was sampled hot, with the engine still running, via the dip-stick. Oil from a single dip of the dip-stick was transferred to both an unwashed and washed 3:1 Al(OH)₃:cellulose powder matrix pellet pressed at 500 kg/sq in. Duplicate pellets (without oil) were prepared as blanks and all four pellets analysed by UV LA-ICP-MS. Instrument settings as for Experiment 5 were used, with minor adjustments for day-to-day variations. The results of analysis are presented in Appendix Experiment 13.

When blank corrected, there is very little difference between results obtained on the unwashed and washed matrices. If the two matrices are treated as a single matrix, then precisions, with the exception of iron, are excellent, commonly being <1 for the restricted range of analytes expected in oil. Reproducibility of the data, are thus excellent and this is graphically illustrated in the X-Y log plot of ‘concentration’ versus elements comprising Chart Experiment 13/1. Here, consistent with the precision/reproducibility data, iron excepted, the two profiles are effectively superimposed upon each other.

The experiment clearly indicates the general reproducibility of the analysis and indicates considerable promise for the technique.

Experiment 15

This experiment had as its main objective, the analysis of oil from the engines of five different cars, collected under the same conditions as described above, that is hot with the engines running, on three consecutive days, to assess whether contrasts in wear metal content in oil form cars of contrasting age, engine capacity and, presumably oil used, could be established. For one ‘old’ car, which required frequent all top-ups between services, a sample of the new top-up oil was available for comparison. The oil was collected as for Experiment 13, but in duplicate on unwashed 3:1 Al(OH)₃:cellulose powder pellets pressed at 100 kg/sq in pressure; new reference oil was dipped with a glass rod and applied, in duplicate, to equivalent pellets. All samples were analysed by UV LA-ICP-MS; the results of the expanded range of analytes are presented as Appendix Experiment 15.

During the course of the analysis, eleven glass standard measurements were made. The precisions on the raw glass data are generally in the range 10 to 20%. However, when the raw data are normalised to average cerium, precisions are generally excellent and, with the exception of selenium, cadmium and mercury, are <10; selenium and cadmium are just marginally higher and mercury sits at 24%. The cerium normalised glass standard data have been plotted in a log X-Y line chart plot which comprises Chart Experiment 15/1. Here, it is clear that the several profiles essentially superimpose, consistent with the very good precisions and reproducibility. In addition to the glass standard, 10 air blank measurements were made throughout the analytical run. These have been drift corrected and the average drift corrected air blank has been used to correct the reported data.

Assessment of the data clearly demonstrates significant, and often marked differences, in specific analytes between the engine oils from the different vehicles. Oil from two cars, ‘John’ and ‘Scott’, were selected to demonstrate these contrasts. ‘John’ engine oil is plotted as a log X-Y line chart in Chart Experiment 15/2 while ‘Scott’ oil comprises Chart Experiment 15/3. Examination of the respective Charts illustrates that while, there is general profile superimposition for the respective replicate oil analyses, there are some clear difference in the shapes of the respective profiles as well as peak height contrasts between equivalent analytes. Chart Experiment 15/4 graphs the averaged composition of ‘John’ and ‘Scott’ oil (n=8). This latter Chart clearly emphasises the marked compositional contrast between the two oils. Hence, from this experiment, it may reasonably be concluded that the technique can readily identify and measure analyte contrasts in the examined engine oils. It is clear from the pilot experiments that wear metal analysis of oils of plant in service by LA-ICP-MS techniques is feasible and useful. The experimentation into the analysis of wear metals in oils indicates considerable potential economic benefits of being able to, for example, regularly monitor potential component wear, through ‘dip-stick’ sampling, in plant in service, that is without the need to plant take off-line, are large. In this way plant down-time can be carefully scheduled with minimal impact upon operations.

The use of a defocused laser to ablate sample matrices is a variation of the protocols described, which can be used to improve laser coupling to the sample. If a laser is focused on the surface of a sample, the first crater it produces is a response to the laser focal point being on the surface of the sample. As soon as the surface material has been ablated and removed, the next ablation event (laser shot) is into the crater area from the first shot where there is no focus and, therefore, the laser coupling is diminished. If, however, the laser is focused below the surface, that is, it is defocused at the surface, potentially it is now possible to generate a more active ablation because a large amount of material can be ejected from the middle of the sample because the focussing is below the surface. Hence, it might be expected that at least the first and second shots will produce a lot of ablation debris and therefore this may increase the sensitivity because, at this stage the ablation ejects is a powder/aerosol and this may be more efficiently transported to the plasma torch. For the existing equipment, laser defocusing can be fairly readily achieved manually. Modern lasers have automatic defocus capabilities where the depth for defocusing can be simply programmed.

As a further modification of the present protocols, triple shot ablation, as compared with double shot, at each point in a 10 point by 10 point raster grid, may be used.

### Example 7

### Quantitation Using Solution Doped Matrices (Further Experiments)

In this example three fibrous cellulose matrices, being Whatman 541, high purity Whatman 541 and old Whatman 540 filter papers (Whatman International Ltd, Maidstone, England), were prepared as blank material by affixing to a support substrate using a backing tape; a sample of the backing tape (3M Scotch Permanent Double Stick Tape) was also analysed. The raw count data was analysed firstly as isotopic concentrations for the designated elements and secondly as elemental abundance concentrations derived from the isotopic data using natural abundance relations. All elemental data has been air blank corrected. Air blank correction has produced negative values for isolated analytes implying that the analyte concentrations in the average air blank are significantly higher than in the matrices for those analytes. Examination of the data illustrates generally high analyte air blank values.

All elements have been spike corrected (ie. normalised to an average value for the spike) and ‘old’ refers to fibrous cellulose substrates that have previously been opened and exposed to the laboratory environment through ‘open’ long-term storage. ‘New’ refers to sealed fibrous cellulose substrates opened for this experiment. With respect to the single versus multiple layer substrate data, it appears probable that analysis of single layer substrates may have involved laser penetration into the backing tape. Hence, data for single layer substrates may reflect composite data whereas for the multiple layers, where the top layer was peeled off immediately prior to analysis, the data reflect only the cellulose matrix substrate.

The data illustrated lower concentrations for a significant number of analytes in multiple, relative to single, layer matrices; other analytes are essentially equivalent while some are higher. For many analytes, for example Cu, Zn, Sn, concentrations in the backing tape is very much greater than in the both the single and multi layer matrices but, here, the single layer matrices are much higher in these elements than the equivalent multi layer material. This strongly suggests that laser penetration to the backing tape has occurred and that much of the difference between single and multi layers has little to do with handling contamination.

Furthermore, the corresponding data for ‘new’ versus ‘old’ clearly demonstrates significantly lower overall concentrations in the new matrices, both single and multiple. This latter observation strongly suggests that long-term exposure of matrices to the laboratory environment has led to variable, but significant ambient laboratory contamination of exposed matrices.

Further experiments examined white and black Whatman 540 filter paper cellulose matrices (Whatman International Ltd, Maidstone, England) doped with 1 ppm multi-element standard (details are provided in the table) and with blood.

The data have been matrix blank corrected. For many of the analytes the air blank is high and similar to the concentrations measured in the white and black cellulose blanks (matrices without samples applied).

The isotopic data, as obtained, was converted to elemental concentrations and the multi element standard and blood doped samples have effectively been doubly corrected. The respective white and black cellulose matrix blanks have first been air blank corrected using the average of two air blanks. Following this, the averaged data, for multi standard and blood doped white and black cellulose, have been corrected using the respective corrected air blank corrected white and black cellulose matrix blanks. There is good correlation between the averaged corrected values for white and black multi element standard doped matrix samples and white and black blood doped samples. Little difference exists between the multi element standard and the blood on white and black matrices. The data obtained in this experiment also illustrates excellent reproducibility for the vast majority of analyst across the mass spectrum in both multi element and blood doped matrices.

Comparison of the computed concentrations in the blood may now be compared with anticipated concentration ranges from the literature. Data for Fe, Cu Zn, Sn, Ba and Pb show very good agreement.

Hardware Optimisation

This experiment was to evaluate hardware optimisation at low, medium and high mass, using respectively manganese, lanthanum and lead. The isotopic data (isotopic concentrations), as obtained, has been rearranged and treated in a manner analogous to that in Example 7. For the current data, air blank, 540 matrix blank, 1 ppm multi element standard and blood doped matrices were examined during optimisation at the relevant masses. Again, the respective 540 matrix blanks have been air blank corrected by subtracting the averaged values from the averaged matrix blank values. Using the corrected matrix blanks, both the 540 multi element and blood doped matrices have been matrix corrected. Again using the corrected date, concentrations in ppb in blood have been computed.

The current data appear to indicate that low mass optimisation may be preferable. When doubly corrected, the indications are that, both for the multi element and blood doped matrices, optimisation at the lower mass, that is manganese, appears preferable to the mid mass and to the high mass. Once again, it is clear, with respect to quantification of trace element in the blood, matrix matched standards are of particular value.

Detection Limits and Precision

The experiment was designed to establish detection limits, precision and quantitation for solution doped cellulose matrices. A series of standards were used for these experiments. In addition a reagent blank was also used.

Deionised water samples were doped, using a ‘stock’ multi-element standard solution, to produce a series of aqueous multi-element standard solutions with element concentrations of 100, 200; 500; 1000; 2000; 5000 and 10000 ppb, 100 μL of each of these aqueous standard solutions was transferred to fibrous cellulose matrix pads, prepared from Whatman 540 filter paper (Whatman International Ltd, Maidstone, England), using a pipette; the pads were affixed to Perspex supports using 3M Scotch Permanent Double Stick Tape. Deionised water matrix blanks were also prepared by pipetting 100 μL of deionised water onto the matrix pads. In addition, solutions of three Certified Reference Materials, SARM's 1, 3 and 46 (South African Bureau of Standards) were diluted 250 times, and 100 μL aliquots of each were doped onto Whatman 540 matrix pads. In all, 10 matrix pads of each aqueous standard concentration and CRM were prepared along with deionised water matrix blanks. A 2 ppm samarium internal standard solution spike was added to the respective matrix pads to facilitate internal normalisation; the spike was added using a pipette. All doped matrix pads were dried at 105° C. for two hours prior to ablation.

Five of each set of ten prepared matrices were analysed on successive days. The sample holders, with affixed matrix pads, were pieced in the laser ablation cell of a UP 266 UV Laser System connected to an X Series ICP-MS with Xi Cone System (Thermo Optek (Australia) Pty Ltd. Rydalmere, Australia) and ablated on a 10×10 matrix raster using a UV laser operating at 266 nm, 10 Hz at a fluence of 6 Milljoule and an argon flow between 900 and 1000 mL per minute for 60 seconds.

Samples were analysed manually and results have been corrected for air blanks, facilitating cross comparison between CRM and standard matrix matched samples. The output data was acquired as raw counts from on-board software and exported into Excel and manipulated. No algorithms were used for computations. From these corrected data, Standard Deviations and Coefficients of Variation have been computed as measures of reproducibility and precision. Finally, quantitative trace element compositions for the 44 analytes examined in the exemplary run were computed for the CRM's; sub-20 ppb detection limits for most analytes were achieved.

Data obtained data is set out in Appendix Experiment M1. It is also quite apparent that data for the standards, when plotted, indicate excellent calibration can be achieved. Quantitation of data for the CRM's indicated extremely good agreement for elemental concentrations for all elements with values (for samples once diluted) in the optimum analytical range of the technique.

There are a number of points that this data demonstrates,
1) It is possible to achieve sub 5% precision for a wide range of elements using the analytical protocols developed in conjunction with ICP-MS.
2) It is possible to achieve sub 20 ppb detection limits for a wide range of elements simultaneously.
3) It is possible to achieve accurate quantitative data, using matrix matched certified reference materials, or other equivalent CRM's.

Examples of useful areas of application of the methods and devices of the present invention are:
screening occupationally exposed workers for anomalous levels of a range of toxic metals;
monitoring environmental exposure of the general population to toxic metals;
screening populations for trace/ultra trace element deficiencies for preventative medicine
screening trace/ultra trace element deficiencies, and toxic heavy metal excesses, in bloodstock, general livestock, zoo animals (including animals in endangered species breeding programs), and domestic pets for veterinary medicine; and monitoring heavy metal pollutants in slaughter animals for meat product quality control in the human food chain.
Monitoring/detecting wear of mechanical components of plant, machinery and the like by analysing lubricating oils.

Although the invention has been described with reference to certain preferred embodiments, variations in keeping with the broad principles and the spirit of the invention are also contemplated as being within its scope.

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

We claim:

1. Sample collection device comprising an inert collection matrix capable of adsorbing or absorbing a fluid sample, and a solid support, wherein the inert matrix is affixed to an area of the solid support.

2. A device according to claims 1, wherein the collection matrix is selected from the group consisting of aragonite, aluminium hydroxide, titania, glucose, Starch “A”, Starch “B”, glucodin, cellulose powder/granules, fibrous cellulose, hydroxy butyl methyl cellulose, vegetable flour or mixtures thereof

3. A device according to claims 2, wherein the vegetable flour is selected from the group consisting of rice, maize, wheat, soy, rye and corn flour, or mixtures thereof.

4. A device according to any one of the preceding claims, wherein the collection matrix is fibrous cellulose.

5. A device according to claim 4, wherein the fibrous cellulose matrix is modified by oxidation and/or acid hydrolysis.

6. A device according to any one of the preceding claims, further comprising, on or within the matrix, one or more pre-calibrated selected analytes as internal standard.

7. A device according to claim 6 wherein the pre-calibrated analytes are represented by or selected from the sets:
Li, Na, Mg, Al, P, K, Ca, Ti, V, Cr, Mn, Fe, Co, Ni, Cu, Zn, Ga, As, Se, Rb, Sr, Mo, Cd, Sn, Sb, Te, Ba, La, Ce, Eu, Dy, Yb, Mg, Tl, Pb, Bi, Th and U;
Li, B, Mg, Al, Si, P, Ca, Ti, V, Cr, Mn, Fe, Co, Ni, Cu, Zn, Ga, As, Se, Sr, Y, Zr, Mo, Ag, Cd, Sn, Sb, Ba, La, Ce, Hf, Hg, Pb and U or
Li, Na, Mg, Al, P, K, Ca, Ti, V, Cr, Mn, Fe, Co, Ni, Cu, Zn, Ga, As, Se, Rb, Sr, Mo, Cd, Sn, Sb, Te, Ba, La, Ce, Eu, Dy, Yb, Hg, Tl, Pb, Bi, Th and U.

8. A device according to any one of the preceding claims, further comprising a test sample.

9. A device according to claim 8, wherein the support comprises a bar-code incorporating information on the sample.

10. A device according to any one of the preceding claims, further comprising an integral lancing member, capable of piercing skin or tissue, to aid in the collection and application of a sample to the inert matrix.

11. A device according to claim 10, wherein the lancing member is mounted adjacent to, within or below the area of inert matrix.

12. A device according to claim 10 or claim 11, further comprising a guiding channel in the inert matrix, to guide the lance when the lance is disposed below the inert matrix area.

13. A device according to any one of the preceding claims, further comprising an integral or separate cover sheath, which covers the matrix.

14. A sample collection device having multi-layer construction wherein the collection matrix layer is sandwiched between two supporting layers, one of said supporting layers having an opening, which exposes an area of the collection matrix.

15. A device according to any one of the preceding claims, wherein the sample is a fluid sample selected from body fluids, oils and water.

16. A device according to claim 15, wherein the body fluid is selected from whole blood, urine and sweat.

17. Method of detecting simultaneously a plurality of elements in a fluid sample adsorbed onto or into an inert collection matrix, comprising:
(i) exposing the sample to high energy radiation capable of ionising at least a portion of the sample, and
(ii) detecting plurality of elements in the ionised portion of the sample by mass spectrometry.

18. Method of quantifying simultaneously a plurality of elements in a fluid sample adsorbed onto or into an inert collection matrix, comprising:
(i) exposing the sample to high energy radiation capable of ionising at least a portion of the sample;
(ii) measuring quantity of a plurality of elements in the ionised portion of the sample by mass spectrometry;
(iii) measuring quantity of ionised portion of sample, and
(iv) determining quantity of the plurality of elements in the sample with reference to the quantity of ionised portion of the sample.

19. Method of quantifying simultaneously a plurality of elements in a fluid sample adsorbed onto or into an inert collection matrix having an internal standard applied thereto, comprising:
(i) exposing the sample to high energy radiation capable of ionising at least a portion of the sample and a portion of said internal standard;
(ii) measuring quantity of a plurality of elements in the ionised portion of the sample by mass spectrometry;
(iii) measuring quantity of ionised internal standard in the ionised portion of the sample by mass spectrometry, and
(iv) determining quantity of the plurality of elements in the sample with reference to quantity of ionised internal standard.

20. Method of quantifying simultaneously a plurality of elements in a fluid sample adsorbed onto an inert collection matrix, comprising:
(i) introducing into the fluid sample a known quantity of a measurable internal standard
(ii) exposing the sample to high energy radiation capable of ionising at least a portion of the sample and the internal standard;
(iii) measuring quantity of a plurality of elements in the ionised portion of the sample by mass spectrometry;
(iv) measuring quantity of ionised internal standard in the ionised portion of the sample by mass spectrometry, and
(v) determining quantity of the plurality of elements in the sample with reference to quantity of ionised internal standard.

21. Method of quantifying simultaneously a plurality of elements in a fluid sample adsorbed/absorbed onto or into an inert collection matrix comprising:
(i) exposing the sample to high energy radiation capable of ionising at least a portion of the sample;
(ii) measuring quantity of a plurality of elements in the ionised portion of the sample by mass spectrometry;
(iii) exposing a matrix-matched Certified Reference Material (CRM) to high energy radiation capable of ionising at least a portion of the CRM;
(iv) measuring quantity of ionised CRM in the ionised portion of the sample by mass spectrometry, and
(v) determining quantity of the plurality of elements in the sample with reference to the CRM.

22. Method of quantifying simultaneously a plurality of elements in a fluid sample supported on an impermeable substrate, comprising:
(i) exposing the sample to high energy radiation capable of ionising at least a portion of the sample;
(ii) measuring quantity of a plurality of elements in the ionised portion of the sample by mass spectrometry;
(iii) exposing a matrix-matched Certified Reference Material (CRM) to high energy radiation capable of ionising at least a portion of the CRM;
(iv) measuring quantity of ionised CRM in the ionised portion of the sample by mass spectrometry, and
(v) determining quantity of the plurality of elements in the sample with reference to the CRM.

23. A method according to claim 19 or claim 20, wherein the internal standard is selected from the group consisting of Li, Na, Mg, Al, P, K, Ca, Ti, V, Cr, Mn, Fe, Co, Ni, Cu, Zn, Ga, As, Se, Rb, Sr, Mo, Cd, Sn, Sb, Te, Ba, La, Ce, Eu, Dy, Yb, Hg, Tl, Pb, Bi, Th and U.

24. A method according to claim 19 or claim 20, wherein the internal standard is selected from the sets:
Li, Na, Mg, Al, P, K, Ca, Ti, V, Cr, Mn, Fe, Co, Ni, Cu, Zn, Ga, As, Se, Rb, Sr, Mo, Cd, Sn, Sb, Te, Ba, La, Ce, Eu, Dy, Yb, Hg, Tl, Pb, Bi, Th and U;
Li, B, Mg, Al, Si, P, Ca, Ti, V, Cr, Mn, Fe, Co, Ni, Cu, Zn, Ga, As, Se, Sr, Y, Zr, Mo, Ag, Cd, Sn, Sb, Ba, La, Ce, Hf, Hg, Pb and U or
Li, Na, Mg, Al, P, K, Ca, Ti, V, Cr, Mn, Fe, Co, Ni, Cu, Zn, Ga, As, Se, Rb, Sr, Mo, Cd, Sn, Sb, Te, Ba, La, Ce, Eu, Dy, Yb, Hg, Tl, Pb, Bi, Th and U.

25. A method according to claim 21 or claim 22, wherein the CRM is selected from the group consisting of SARM 1, 3 and 48, and SY-2.

26. A method according to any one of claims 17 to 24, wherein the inert collection matrix is part of a sample collection device according to any one of claim 1 to 14.

27. A method according to any one of claims 17 to 26, wherein the fluid sample is selected from body fluids, oils and water.

28. A method according to claim 27, wherein the body fluid is selected from whole blood, urine and sweat.

29. A method according to claim 28, wherein the sample is whole blood and sample size is about 50 μl to about 100 μl.

30. A method according to claim 28, wherein the sample size is about 50 μl or less.

31. A method according to any one of claims 17 to 30, wherein the high energy radiation is UV laser radiation.

32. A method according to claim 31, wherein the laser radiation is a component of Inductively Coupled Plasma-Mass Spectrometer (ICP-MS).

33. A method according to claim 32, wherein the mass spectrometer is selected from quadrupole and Time-of-Flight (TOF).

34. A method according to any one of claims 17 to 33, wherein the sample is exposed to radiation for a period of from about 10 seconds to about 120 seconds.

35. A method according to any one of claims 17 to 34, wherein the elements to be detected and/or quantified are selected from dietary trace elements, toxic elements and markers of pollution or wear and tear.

36. A method according to any one of claims 17 to 34, wherein the matrix or the support comprise one or more wells or indentations to accommodate the fluid sample.

37. A method of collecting a fluid sample for mass spectrometry analysis of multiple element content comprising the application of the sample to an inert matrix having a low background element content, wherein the matrix is selected from the group consisting of aragonite, aluminium hydroxide, titania, glucose, Starch “A”, Starch “B”, glucodin, cellulose powder/granules, fibrous cellulose, hydroxy butyl methyl cellulose, vegetable flour or mixtures thereof.
